SEQUENCE LISTING (34 sequences)

SEQ ID NO: 1 - protein, 15 aa, Artificial sequence
  Pembrolizumab-Light chain CDR1
RASKGVSTSGYSYLH

SEQ ID NO: 2 - protein, 7 aa, Artificial Sequence
  Pembrolizumab-Light chain CDR2
LASYLES

SEQ ID NO: 3 - protein, 9 aa, Artificial Sequence
  Pembrolizumab-Light chain CDR3
QHSRDLPLT

SEQ ID NO: 4 - protein, 111 aa, Artificial Sequence [sequence omitted]

SEQ ID NO: 5 - protein, 218 aa, Artificial Sequence
  Pembrolizumab-Light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Pembrolizumab-Heavy chain CDR1
NYYMY

SEQ ID NO: 7 - protein, 17 aa, Artificial sequence
  Pembrolizumab-Heavy chain CDR2
GINPSNGGTNFNEKFKN

SEQ ID NO: 8 - protein, 11 aa, Artificial sequence
  Pembrolizumab-Heavy chain CDR3
RDYRFDMGFDY

SEQ ID NO: 9 - protein, 120 aa, Artificial sequence [sequence omitted]

SEQ ID NO: 10 - protein, 447 aa, Artificial sequence
  Pembrolizumab-Heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, Homo sapiens
RASQSVSSYLA

SEQ ID NO: 12 - protein, 7 aa, Homo sapiens
DASNRAT

SEQ ID NO: 13 - protein, 9 aa, Homo sapiens
QQSSNWPRT

SEQ ID NO: 14 - protein, 107 aa, Homo sapiens [sequence omitted]

SEQ ID NO: 15 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, Homo sapiens
NSGMH

SEQ ID NO: 17 - protein, 17 aa, Homo sapiens
VIWYDGSKRYYADSVKG

SEQ ID NO: 18 - protein, 4 aa, Homo sapiens
NDDY

SEQ ID NO: 19 - protein, 113 aa, Homo sapiens [sequence omitted]

SEQ ID NO: 20 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 15 aa, Artificial sequence
  hPD-1.08A Light Chain CDR1
RASKSVSTSGFSYLH

SEQ ID NO: 22 - protein, 7 aa, Artificial sequence
  hPD-1.08A Light Chain CDR2
LASNLES

SEQ ID NO: 23 - protein, 9 aa, Artificial sequence
  hPD-1.08A Light Chain CDR3
QHSWELPLT

SEQ ID NO: 24 - protein, 5 aa, Artificial sequence
  hPD-1.08A Heavy Chain CDR1
SYYLY

SEQ ID NO: 25 - protein, 17 aa, Artificial sequence
  hPD-1.08A Heavy Chain CDR2
GVNPSNGGTNFSEKFKS

SEQ ID NO: 26 - protein, 11 aa, Artificial sequence
  hPD-1.08A Heavy Chain CDR3
RDSNYDGGFDY

SEQ ID NO: 27 - protein, 120 aa, Artificial sequence [sequence omitted]

SEQ ID NO: 28 - protein, 111 aa, Artificial sequence [sequence omitted]

SEQ ID NO: 29 - protein, 111 aa, Artificial sequence
  K09A light chain variable region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 30 - protein, 111 aa, Artificial sequence
  K09A light chain variable region
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 447 aa, Artificial sequence; identical to SEQ ID NO: 10
  mature 409 heavy chain

SEQ ID NO: 32 - protein, 218 aa, Artificial sequence; identical to SEQ ID NO: 5
  mature K09A light chain

SEQ ID NO: 33 - protein, 218 aa, Artificial sequence
  mature K09A light chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, Artificial sequence
  mature K09A light chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0112564 A1
(43) Pub. Date: Apr. 14, 2022

(54) GENE EXPRESSION BASED BIOMARKER OF TUMOR RESPONSE TO PD-1 ANTAGONISTS

(71) Applicants: Andrey LOBODA, Boston, MA (US); Jared K. LUNCEFORD, Washington, UT (US); Chunsheng ZHANG, Boston, MA (US); Michael NEBOZHYN, West Point, PA (US); Razvan CRISTESCU, Boston, MA (US); Mark D. AYERS, West Point, PA (US); Terrill K. MCCLANAHAN, South San Francisco, CA (US); Hua MA, North Wales, PA (US); Merck Sharp & Dohme Corp., Rahway, NJ (US)

(72) Inventors: Andrey Loboda, Canton, MA (US); Jared K. Lunceford, Washington, UT (US); Chunsheng Zhang, Walpole, MA (US); Michael Nebozhyn, Colmar, PA (US); Razvan Cristescu, Newton, MA (US); Mark D. Ayers, Pennington, NJ (US); Terrill K. McClanahan, Sunnyvale, CA (US); Hua Ma, North Wales, PA (US)

(73) Assignee: Merck Sharp & Dohme Corp., Rahway, NJ (US)

(21) Appl. No.: 17/430,448

(22) PCT Filed: Feb. 10, 2020
(86) PCT No.: PCT/US2020/017408

§ 371 Date: Aug. 12, 2021

Related U.S. Application Data
(60) Provisional application No. 62/805,560, filed on Feb. 14, 2019.

(51) Int. Cl. C12Q 1/6886; C07K 16/28; A61P 35/00

(52) U.S. Cl. CPC C12Q 1/6886; C07K 16/2818; A61P 35/00; A61K 2039/505; C12Q 2600/106; C12Q 2600/112; C12Q 2600/158; C12Q 2600/16

(57) ABSTRACT

The invention relates to a stromal/EMT/TGFβ signature that is predictive of patient response to treatment with a PD-1 antagonist, wherein the stromal/EMT/TGFβ signature comprises five or more genes selected from Table 1 disclosed herein. More specifically, a lower stromal/EMT/TGF-β score is associated with favorable response to a PD-1 antagonist in a patient with cancer. Also provided are methods of treating a cancer patient with a PD-1 antagonist that were identified as positive for the stromal/EMT/TGFβ biomarker of the invention. The disclosure also provides methods and kits for testing tumor samples for the biomarkers.

### FIELD OF THE INVENTION

The present invention relates generally to the treatment of cancer with antagonists of Programmed Death 1 (PD-1). In particular, the invention relates to identifying patients who are most likely to respond to therapy with a PD-1 antagonist by determining if they are positive or negative for a stromal/EMT/TGFβ gene signature biomarker.

### CROSS-REFERENCE TO RELATED APPLICATIONS

This application claims the benefit of U.S. Provisional Application No. 62/805,560, filed on Feb. 14, 2019, the contents of which are hereby incorporated by reference in their entirety.

### REFERENCE TO SEQUENCE LISTING SUBMITTED ELECTRONICALLY

The sequence listing of the present application is submitted electronically via EFS-Web as an ASCII formatted sequence listing with a file name “247040WPCT-SEQLIST-28JAN2020.TXT”, creation date of Jan. 28, 2020, and a size of 34 kb. This sequence listing submitted via EFS-Web is part of the specification and is herein incorporated by reference in its entirety.

### BACKGROUND OF THE INVENTION

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (Sharpe et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong et al., Nat Med. 8(8):793-800 (2002); Yang et al. Invest Ophthalmol Vis Sci. 49: 2518-2525 (2008); Ghebeh et al. Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953 (2005); Inman et al., Cancer 109: 1499-1505 (2007); Shimauchi et al. Int. J. Cancer 121:2585-2590 (2007); Gao et al. Clin. Cancer Research 15: 971-979 (2009); Nakanishi J. Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T cells in breast cancer and melanoma (Ghebeh et al, BMC Cancer. 2008 8:5714-15 (2008); Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T cells to attenuate T cell activation and evasion of immune surveillance, thereby contributing to an impaired immune response against the tumor.

Immune checkpoint therapies targeting the PD-1 axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33). Immune therapies targeting the PD-1 axis include monoclonal antibodies directed to the PD-1 receptor (KEYTRUDA™ (pembrolizumab), Merck and Co., Inc., Kenilworth, N.J., USA and OPDIVO™ (nivolumab), Bristol-Myers Squibb Company, Princeton, N.J., USA) and also those that bind to the PD-L1 ligand (MPDL3280A; TECENTRIQ™ (atezolizumab), Genentech, San Francisco, Calif., USA; IMFINZI™ (durvalumab), AstraZeneca Pharmaceuticals LP, Wilmington, Del.; BAVENCIO™ (avelumab), Merck KGaA, Darmstadt, Germany). Both therapeutic approaches have demonstrated anti-tumor effects in numerous cancer types.

Although PD-1 antagonists can induce durable anti-tumor responses in some patients in certain cancer types, a significant number of patients fail to respond to therapies targeting PD-1/PD-L1. Thus, a need exists for diagnostic tools to identify which cancer patients are most likely to achieve a clinical benefit to treatment with a PD-1 antagonist. An active area in cancer research is the identification of intratumoral expression patterns for sets of genes, commonly referred to as gene signatures or molecular signatures, which are characteristic of particular types or subtypes of cancer, and which may be associated with clinical outcomes. PD-L1 immunohistochemistry and gene expression profiles (GEP) are associated with response to PD-1/PD-L1 inhibitor therapies in multiple tumor types (McDermott et al. Nat Med. 24:749-757 (2018); Ayers et al. J Clin Invest. 127:2930-2940 (2017); O'Donnell et al. J Clin Oncol. 35: 4502 (2017)). An 18-gene GEP was shown to be associated with a pan tumor response to pembrolizumab (Ayers et al., supra). A biomarker study of patients with cisplatin-ineligible advanced urothelial cancer who were enrolled in clinical trial Keynote-052 also showed that GEP was associated with response to pembrolizumab (O'Donnell et al., supra).

### SUMMARY OF THE INVENTION

The invention relates to a method for testing a tumor for the presence or absence of a biomarker that predicts response to treatment with a PD-1 antagonist, which comprises: (a) obtaining a sample from the tumor, measuring the raw RNA expression level in the tumor sample for each gene in a stromal/EMT/TGFβ gene signature; (b) normalizing each of the measured raw RNA expression levels; and (c) calculating the arithmetic mean of the normalized RNA expression levels for each of the genes to generate a score for the stromal/EMT/TGFβ gene signature; wherein the stromal/EMT/TGFβ gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD1; (d) comparing the calculated score to a reference score for the stromal/EMT/TGFβ gene signature; and (e) classifying the tumor as biomarker positive or biomarker negative; wherein if the calculated score is equal to or less than the reference score, then the tumor is classified as biomarker positive, and if the calculated stromal/EMT/TGFβ gene signature score is greater than the reference stromal/EMT/TGFβ gene signature score, then the tumor is classified as biomarker negative.

Also provided herein is a method for treating cancer in a subject having a tumor which comprises administering to the subject a PD-1 antagonist if the tumor is positive for a stromal/EMT/TGFβ gene signature biomarker, or administering to the subject a cancer treatment that does not include a PD-1 antagonist if the tumor is negative for the biomarker; wherein the determination of whether the tumor is positive or negative for the stromal/EMT/TGFβ gene signature biomarker was made using a method as described herein.

The invention further relates to pharmaceutical compositions comprising a PD-1 antagonist for use in a subject who has a tumor that tests positive for a stromal/EMT/TGFβ gene signature biomarker, wherein the stromal/EMT/TGFβ 1 gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD1.

### DESCRIPTION OF THE DRAWINGS

FIG. 1 provides a scatterplot of NanoString versus RNASeq 18-Gene T-cell inflamed gene expression profile. See EXAMPLE 3.

FIGS. 2A and 2B show associations of RNASeq-based 18-gene T-cell inflamed GEP with clinical response. A box plot (FIG. 2A) and ROC curve (FIG. 2B) of RNASeq 18-gene T-cell inflamed GEP by BOR in the total population are provided.

FIG. 3A provides a box plot of stroma/EMT/TGFβ signature by BOR;

FIG. 3B provides an ROC Curve; and FIG. 3C provides a box plot of stroma/EMT/TGFβ signature by BOR using the 18-gene T-cell inflamed GEP tertiles. See EXAMPLE 3.

FIG. 4 provides a scatter plot for stroma/EMT/TGFβ signature score versus RNASeq 18-gene T-cell inflamed GEP score with response status in the total population.

FIG. 5 provides a Kaplan-Meier curve for PFS by stroma/EMT/TGFβ signature level and 18-gene T-cell inflamed GEP status in the total population.

### DETAILED DESCRIPTION OF THE INVENTION

The invention relates to a stromal/EMT/TGFβ signature that is predictive of patient response to treatment with a PD-1 antagonist, wherein the stromal/EMT/TGFβ signature comprises five or more genes selected from Table 1. More specifically, a lower stromal/EMT/TGF-β score is associated with favorable response to a PD-1 antagonist in a patient with cancer.

TABLE 1
Stromal/EMT/TGFβ gene signature.

| Symbol | Locus Link | Accession No. |
|---|---|---|
| TSHZ3 | 57616 | NM_020856 |
| FILIP1L | 11259 | NM_182909 |
| PCOLCE | 5118 | NM_002593 |
| HSPA12B | 116835 | NM_001197327 |
| GGT5 | 2687 | NM_001099782 |
| RUNX1T1 | 862 | NM_001198633 |
| ELTD1 | | NM_022159 |
| AEBP1 | 165 | NM_001129 |
| PODN | 127435 | NM_153703 |
| ITGA11 | 22801 | NM_001004439 |
| MMP2 | 4313 | NM_004530 |
| LRRC32 | 2615 | NM_001128922 |
| COL15A1 | 1306 | NM_001855 |
| FSTL1 | 11167 | NM_007085 |
| KIAA1462 | | NM_001350022 |
| EDNRA | 1909 | NM_001957 |
| ANTXR1 | 84168 | NM_032208 |
| WISP1 | 8840 | NM_001204870 |
| THY1 | 7070 | NM_001311162 |
| ADAMTS2 | 9509 | NM_014244 |
| COL8A1 | 1295 | NM_001850 |
| MXRA8 | 54587 | NM_001282583 |
| THBS2 | 7058 | NM_003247 |
| AXL | 558 | NM_021913 |
| ECM2 | 1842 | NM_001197296 |
| LAMA4 | 3910 | NM_002290 |
| COL1A2 | 1278 | NM_000089 |
| GLT8D2 | 83468 | NM_031302 |
| DCN | 1634 | NM_133505 |
| CRISPLD2 | 83716 | NM_031476 |
| COL5A2 | 1290 | NM_000393 |
| CDH11 | 1009 | NM_001797 |
| GPR124 | | NM_032777 |
| CD248 | 57124 | NM_020404 |
| COL6A3 | 1293 | NM_004369 |
| NID2 | 22795 | NM_007361 |
| COL6A2 | 1292 | NM_001849 |
| HEG1 | 57493 | NM_020733 |
| COL5A1 | 1289 | NM_001278074 |
| VCAN | 1462 | NM_001126336 |
| COL3A1 | 1281 | NM_000090 |
| MSRB3 | 253827 | NM_198080 |
| CD93 | 22918 | NM_012072 |
| CCDC80 | 151887 | NM_199512 |
| OLFML2B | 25903 | NM_001297713 |
| ANGPTL2 | 23452 | NM_012098 |
| OLFML1 | 283298 | NM_198474 |
| FAM26E | | NM_153711 |
| SPARC | 6678 | NM_001309444 |
| FBN1 | 2200 | NM_000138 |
| PDGFRB | 5159 | NM_002609 |

### I. Definitions and Abbreviations

Throughout the detailed description and examples of the invention the following abbreviations will be used:

BOR best overall response

CDR complementarity determining region

CHO Chinese hamster ovary

CPS combined positive score

CR complete response

DFS disease free survival

ECOG Eastern Cooperative Oncology Group

EMT epithelial to mesenchymal transition

FFPE formalin-fixed, paraffin-embedded

FR framework region

GEP gene expression profile

IHC immunohistochemistry or immunohistochemical

irRC immune related response criteria

NCBI National Center for Biotechnology Information

NPV net predictive value

NR not reached

OR overall response

OS overall survival

PD progressive disease

PD-1 programmed death 1

PD-L1 programmed cell death 1 ligand 1

PD-L2 programmed cell death 1 ligand 2

PFS progression free survival

PPV positive predictive value

PR partial response

Q2W one dose every two weeks

Q3W one dose every three weeks

Q4W one dose every four weeks

Q6W one dose every six weeks

RECIST Response Evaluation Criteria in Solid Tumors

ROC receiver operating characteristic

SD stable disease

TGFβ transforming growth factor-β

UC urothelial cancer

VH immunoglobulin heavy chain variable region

VK immunoglobulin kappa light chain variable region

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” when used to modify a numerically defined parameter (e.g., the gene signature score for a gene signature discussed herein, or the dosage of a PD-1 antagonist, or the length of treatment time with a PD-1 antagonist, or the amount of time between treatments with a PD-1 antagonist) means that the parameter may vary by as much as 10% above or below the stated numerical value for that parameter. For example, a gene signature consisting of about 10 genes may have between 9 and 11 genes. Similarly, a reference gene signature score of about 2.462 includes scores of and any score between 2.2158 and 2.708. In certain embodiments, “about” can mean a variation of ±0.1%, ±0.5%, ±1%, ±2%, ±3%, ±4%, ±5%, ±6%, ±7%, ±8%, ±9% or ±10%. When referring to the amount of time between administrations in a therapeutic treatment regimen (i.e., amount of time between administrations of the PD-1 antagonist, e.g. “about 6 weeks,” which is used interchangeably herein with “approximately every six weeks”), “about” refers to the stated time±a variation that can occur due to patient/clinician scheduling and availability around the 6-week target date. For example, “about 6 weeks” can refer to 6 weeks±5 days, 6 weeks±4 days, 6 weeks±3 days, 6 weeks±2 days or 6 weeks±1 day, or may refer to 5 weeks, 2 days through 6 weeks, 5 days.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Treat” or “treating” a cancer, as used herein, means to administer a PD-1 antagonist, e.g. an anti-PD-1 antibody or antigen binding fragment thereof, to a subject having a cancer, or diagnosed with a cancer, to achieve at least one positive therapeutic effect, such as, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, decreasing the number of one or more tumor markers, halting or delaying the growth of a tumor or blood cancer or progression of disease associated with PD-1 binding to its ligands PD-L1 and/or PD-L2 (“PD-1-related disease”) such as cancer, stabilization of PD-1-related disease, inhibiting the growth or survival of tumor cells, eliminating or reducing the size of one or more cancerous lesions or tumors, decreasing the level of one or more tumor markers, ameliorating or abrogating the clinical manifestations of PD-1-related disease, reducing the severity or duration of the clinical symptoms of PD-1-related disease such as cancer, prolonging the survival of a patient relative to the expected survival in a similar untreated patient, and inducing complete or partial remission of a cancerous condition or other PD-1 related disease.

Positive therapeutic effects in cancer can be measured in a number of ways (See, W. A. Weber, J. Nucl. Med. 50:IS-10S (2009)). In some preferred embodiments, response to a PD-1 antagonist is assessed using RECIST 1.1 criteria or irRC. With respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). In some embodiments, the treatment achieved by a therapeutically effective amount is any of partial response (PR), complete response (CR), PFS, DFS, overall response (OR) or OS.

PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the present invention may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

In some embodiments, a gene signature biomarker of the invention predicts whether a subject with a solid tumor is likely to achieve a PR or a CR. The dosage regimen of a therapy described herein that is effective to treat a cancer patient may vary according to factors such as the disease state, age, and weight of the patient, and the ability of the therapy to elicit an anti-cancer response in the subject. While an embodiment of the treatment methods, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of a parental antibody generated in a mouse for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxyl-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5t ed.; NIH Publ. No. 91-3242 (1991); Kabat (1978) Adv. Prot. Chem. 32:1-75; Kabat, et al., (1977) J. Biol. Chem. 252:6609-6616; Chothia et al., (1987) J Mol. Biol. 196:901-917 or Chothia et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). As used herein, the term “framework” or “FR” residues refers to those variable domain residues other than the hypervariable region residues defined herein as CDR residues.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic agent, such as a PD-1 antagonist, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a PD-1 antagonist is assessed using RECIST 1.1 criteria, bidimensional irRC or unidimensional irRC. In some embodiments, an anti-tumor response is any of SD, PR, CR, PFS, DFS. In some embodiments, a gene signature biomarker of the invention predicts whether a subject with a solid tumor is likely to achieve a PR or a CR.

“Bidimensional irRC” refers to the set of criteria described in Wolchok J D, et al. Guidelines for the evaluation of immune therapy activity in solid tumors: immune-related response criteria. Clin Cancer Res. 2009; 15(23):7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin lymphoma, non-Hodgkin lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Particularly preferred cancers that may be treated in accordance with the present invention include those characterized by elevated expression of one or both of PD-L1 and PD-L2 in tested tissue samples.

“CDR” or “CDRs” as used herein means complementarity determining region(s) in an immunoglobulin variable region, generally defined using the Kabat numbering system.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods of the present invention include cytostatic and/or cytotoxic agents.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, if a gene signature score is defined as the composite RNA expression score for a set of genes that consists of a specified list of genes, the skilled artisan will understand that this gene signature score could include the RNA level determined for one or more additional genes, preferably no more than three additional genes, if such inclusion does not materially affect the predictive power.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J Allergy Clin. Immunol. 116:731.

“Non-responder patient” when referring to a specific anti-tumor response to treatment with a PD-1 antagonist, means the patient did not exhibit the specified anti-tumor response.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length.

The term “patient” (alternatively referred to as “subject” or “individual” herein) refers to a mammal (e.g., rat, mouse, dog, cat, rabbit) capable of being treated with the methods and compositions of the invention, most preferably a human. In some embodiments, the patient is an adult patient. In other embodiments, the patient is a pediatric patient.

“PD-1 antagonist” means any chemical compound or biological molecule that blocks binding of PD-L1 to PD-1 and preferably also blocks binding of PD-L2 to PD-1. As a none limiting example, a PD-1 antagonist blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the various aspects and embodiments of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the various aspects and embodiments of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the various aspects and embodiments of the present invention, are described in U.S. Pat. Nos. 7,521,051, 8,008,449, and 8,354,509. Specific anti-human PD-1 mAbs useful as the PD-1 antagonist various aspects and embodiments of the present invention include: pembrolizumab, a humanized IgG4 mAb with the structure described in WHO Drug Information, Vol. 27, No. 2, pages 161-162 (2013), nivolumab (BMS-936558), a human IgG4 mAb with the structure described in WHO Drug Information, Vol. 27, No. 1, pages 68-69 (2013); pidilizumab (CT-011, also known as hBAT or hBAT-1); and the humanized antibodies h409A11, h409A16 and h409A17, which are described in WO 2008/156712.

Additional PD-1 antagonists useful in any of the various aspects and embodiments of the present invention include a pembrolizumab biosimilar or a pembrolizumab variant.

As used herein “pembrolizumab biosimilar” means a biological product that (a) is marketed by an entity other than Merck and Co., Inc., or a subsidiary thereof, and (b) is approved by a regulatory agency in any country for marketing as a pembrolizumab biosimilar. In an embodiment, a pembrolizumab biosimilar comprises a pembrolizumab variant as the drug substance. In an embodiment, a pembrolizumab biosimilar has the same amino acid sequence as pembrolizumab.

As used herein, a “pembrolizumab variant” means a monoclonal antibody which comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

Examples of mAbs that bind to human PD-L1, and useful in any of the various aspects and embodiments of the present invention, are described in WO2013/019906, WO2010/077634 A1 and U.S. Pat. No. 8,383,796. Specific anti-human PD-L1 mAbs useful as the PD-1 antagonist in the various aspects and embodiments of the present invention include atezolizumab, BMS-936559, MEDI4736, avelumab and durvalumab.

Other PD-1 antagonists useful in any of the various aspects and embodiments of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin on molecules that specifically bind to PD-1 are described in WO 2010/027827 and WO 2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

“Probe” as used herein means an oligonucleotide that is capable of specifically hybridizing under stringent hybridization conditions to a transcript expressed by a gene of interest listed in Table 1, and in some preferred embodiments, specifically hybridizes under stringent hybridization conditions to the particular transcript listed in Table 1 for the gene of interest.

“RECIST 1.1 Response Criteria” as used herein means the definitions set forth in Eisenhauer et al., E. A. et al., Eur. J Cancer 45:228-247 (2009) for target lesions or non-target lesions, as appropriate based on the context in which response is being measured.

“Reference T-cell inflamed GEP gene signature score” as used herein means the score for an T-cell inflamed GEP gene signature that has been determined to divide at least the majority of responders from at least the majority of non-responders in a reference population of subjects who have the same tumor type as a test subject and who have been treated with a PD-1 antagonist. Preferably, at least any of 60%, 70%, 80%, or 90% of responders in the reference population will have an T-cell inflamed GEP gene signature nature score that is above the selected reference score, while the T-cell inflamed GEP gene signature score for at least any of 60%, 70% 80%, 90% or 95% of the non-responders in the reference population will be lower than the selected reference score. In some embodiments, the negative predictive value of the reference score is greater than the positive predictive value. In some preferred embodiments, responders in the reference population are defined as subjects who achieved a partial response (PR) or complete response (CR) as measured by RECIST 1.1 criteria and non-responders are defined as not achieving any RECIST 1.1 clinical response. In particularly preferred embodiments, subjects in the reference population were treated with substantially the same anti-PD-1 therapy as that being considered for the test subject, i.e., administration of the same PD-1 antagonist using the same or a substantially similar dosage regimen.

“Sample” when referring to a tumor or any other biological material referenced herein, means a tissue sample that has been removed from the subject's tumor; thus, the testing methods described herein are not performed in or on the subject (although the methods of treatment of the invention clearly include treating the subject).

“Responder patient” when referring to a specific anti-tumor response to treatment with a PD-1 antagonist, means the patient exhibited the anti-tumor response.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC refers to the set of criteria described in Nishino M, Giobbie-Hurder A, Gargano M, Suda M, Ramaiya N H, Hodi F S. Developing a Common Language for Tumor Response to Immunotherapy: Immune-related Response Criteria using Unidimensional measurements. Clin Cancer Res. 2013; 19(14):3936-3943). These criteria utilize the longest diameter (cm) of each lesion.

“Variable regions” or “V region” as used herein means the segment of IgG chains which is variable in sequence between different antibodies. It extends to Kabat residue 109 in the light chain and 113 in the heavy chain.

### II. Methods and Uses of the Invention

In one aspect, the invention relates to a method for testing a tumor for the presence or absence of a biomarker that predicts response to treatment with a PD-1 antagonist, which comprises: (a) obtaining or receiving a sample from the tumor, (b) measuring the raw RNA expression level in the tumor sample for each gene in a stromal/EMT/TGFβ gene signature; (c) normalizing each of the measured raw RNA expression levels; and (d) calculating the arithmetic mean of the normalized RNA expression levels for each of the genes to generate a score for the stromal/EMT/TGFβ gene signature; wherein the stromal/EMT/TGFβ gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD1; (e) comparing the calculated score to a reference score for the stromal/EMT/TGFβ gene signature; and (f) classifying the tumor as biomarker positive or biomarker negative; wherein if the calculated score is equal to or less than the reference score, then the tumor is classified as biomarker positive, and if the calculated stromal/EMT/TGFβ gene signature score is greater than the reference stromal/EMT/TGFβ gene signature score, then the tumor is classified as biomarker negative.

In particular embodiments, the stromal/EMT/TGFβ gene signature comprises at least ten genes selected from the list above (i.e. at least 10 genes selected from Table 1). In other embodiments, the stromal/EMT/TGFβ gene signature comprises at least 11 genes, at least 12 genes, at least 13 genes, at least 14 genes, at least 15 genes, at least 16 genes, at least 17 genes, at least 18 genes, at least 19 genes, at least 20 genes, at least 21 genes, at least 22 genes, at least 23 genes, at least 24 genes, at least 25 genes, at least 26 genes, at least 27 genes, at least 28 genes, at least 29 genes, at least 30 genes, at least 31 genes, at least 32 genes, at least 33 genes, at least 34 genes, at least 35 genes, at least 36 genes, at least 37 genes, at least 38 genes, at least 39 genes, at least 40 genes, at least 41 genes, at least 42 genes, at least 43 genes, at least 44 genes, at least 45 genes, at least 46 genes, at least 47 genes, at least 48 genes, at least 49 genes, at least 50 genes, or 51 genes from Table 1.

In one embodiment, the stromal/EMT/TGFβ gene signature comprises the following genes: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, and ELTD1.

By measuring RNA levels for each gene in Table 1 and then computing signature scores from the normalized RNA levels for only the genes in each gene signature of interest, a single gene expression analysis system may be used to generate and evaluate gene signature scores for different gene signatures and different tumor types to derive candidate biomarkers of anti-tumor response to a PD-1 antagonist.

In particular embodiments, step (b) comprises normalizing each of the measured raw RNA levels for each gene in the stromal/EMT/TGFβ gene signature using the measured RNA levels of a set of normalization genes.

In some embodiments, the set of normalization set comprises 10-12 housekeeping genes.

In particular embodiments, the normalization set comprises the following genes: ABCF1, C14ORF102, G6PD, OAZ1, POLR2A, SDHA, STK11IP, TBC1D10B, TBP, UBB, and ZBTB34.

Gene signature scores may be derived by using the entire clinical response gene set (i.e. all of the genes specified in Table 1), or any subset thereof, as a set of input covariates to multivariate statistical models that will determine signature scores using the fitted model coefficients, for example the linear predictor in a logistic or Cox regression. One specific example of a multivariate strategy is the use of elastic net modeling (Zou & Hastie, 2005, J. R. Statist Soc. B, 67(2): 301-320; Simon et al., 2011, J. Statistical Software 39(5): 1-13), which is a penalized regression approach that uses a hybrid between the penalties of the lasso and ridge regression, with cross-validation to select the penalty parameters. Because the RNA expression levels for most, if not all, of the clinical response genes are expected to be predictive, in one embodiment the L1 penalty parameter may be set very low, effectively running a ridge regression.

A multivariate approach may use a meta-analysis that combines data across cancer indications or may be applied within a single cancer indication. In either case, analyses would use the normalized intra-tumoral RNA expression levels of the signature gene as the input predictors, with anti-tumor response as the dependent variable. The result of such an analysis algorithmically defines the signature score for tumor samples from the patients used in the model fit, as well as for tumor samples from future patients, as a numeric combination of the multiplication co-efficients for the normalized RNA expression levels of the signature genes that is expected to be predictive of anti-tumor response. The gene signature score is determined by the linear combination of the signature genes, as dictated by the final estimated values of the elastic net model coefficients at the selected values of the tuning parameters. Specifically, for a given tumor sample, the estimated coefficient for each gene is multiplied by the normalized RNA expression level of that gene in the tumor sample and then the resulting products are summed to yield the signature score for that tumor sample. Multivariate model-based strategies other than elastic net could also be used to determine a gene signature score.

An alternative to such model-based signature scores would be to use a simple averaging approach, e.g., the signature score for each tumor sample would be defined as the average of that sample's normalized RNA expression levels for those signature genes deemed to be positively associated with the anti-tumor response minus the average of that sample's normalized RNA expression levels for those signature genes deemed to be negatively associated with the anti-tumor response.

### Utility of Gene Signatures and Biomarkers of the Invention

The stromal/EMT/TGFβ gene signature biomarker may be useful to identify cancer patients who are most likely to achieve a clinical benefit from treatment with a PD-1 antagonist. This utility supports the use of such biomarkers in a variety of research and commercial applications, including but not limited to, clinical trials of PD-1 antagonists in which patients are selected on the basis of whether they test positive or negative for a gene signature biomarker, diagnostic methods and products for determining a patient's gene signature score or for classifying a patient as positive or negative for a gene signature biomarker, personalized treatment methods which involve tailoring a patient's drug therapy based on the patient's gene signature score or biomarker status, as well as pharmaceutical compositions and drug products comprising a PD-1 antagonist for use in treating patients who test positive for a gene signature biomarker.

The utility of any of the research and commercial applications claimed herein does not require that 100% of the patients who test positive for a gene signature biomarker achieve an anti-tumor response to a PD-1 antagonist; nor does it require a diagnostic method or kit to have a specific degree of specificity or sensitivity in determining the presence or absence of a biomarker in every subject, nor does it require that a diagnostic method claimed herein be 100% accurate in predicting for every subject whether the subject is likely to have a beneficial response to a PD-1 antagonist. Thus, it is intended that the terms “determine”, “determining” and “predicting” should not be interpreted as requiring a definite or certain result; instead these terms should be construed as meaning either that a claimed method provides an accurate result for at least the majority of subjects or that the result or prediction for any given subject is more likely to be correct than incorrect.

Preferably, the accuracy of the result provided by a diagnostic method of the invention is one that a skilled artisan or regulatory authority would consider suitable for the particular application in which the method is used. Similarly, the utility of the claimed drug products and treatment methods does not require that the claimed or desired effect is produced in every cancer patient; all that is required is that a clinical practitioner, when applying his or her professional judgment consistent with all applicable norms, decides that the chance of achieving the claimed effect of treating a given patient according to the claimed method or with the claimed composition or drug product.

### Assaying Tumor Samples for Gene Signatures and Biomarkers

A gene signature score is determined in a sample of tumor tissue removed from a subject. The tumor may be primary or recurrent, and may be of any type (as described above), any stage (e.g., Stage I, II, III, or IV or an equivalent of other staging system), and/or histology. The subject may be of any age, gender, treatment history and/or extent and duration of remission.

The tumor sample can be obtained by a variety of procedures including, but not limited to, surgical excision, aspiration or biopsy. The tissue sample may be sectioned and assayed as a fresh specimen; alternatively, the tissue sample may be frozen for further sectioning. In some preferred embodiments, the tissue sample is preserved by fixing and embedding in paraffin or the like.

The tumor tissue sample may be fixed by conventional methodology, with the length of fixation depending on the size of the tissue sample and the fixative used. Neutral buffered formalin, glutaraldehyde, Bouin's and paraformaldehyde are non-limiting examples of fixatives. In preferred embodiments, the tissue sample is fixed with formalin. In some embodiments, the fixed tissue sample is also embedded in paraffin to prepare an FFPE tissue sample.

Typically, the tissue sample is fixed and dehydrated through an ascending series of alcohols, infiltrated and embedded with paraffin or other sectioning media so that the tissue sample may be sectioned. Alternatively, the tumor tissue sample is first sectioned and then the individual sections are fixed.

In some preferred embodiments, the gene signature score for a tumor is determined using FFPE tissue sections of about 3-4 millimeters, and preferably 4 micrometers, which are mounted and dried on a microscope slide.

Once a suitable sample of tumor tissue has been obtained, it is analyzed to quantitate the RNA expression level for each of the genes in Table 1, or for a gene signature derived therefrom. The phrase “determine the RNA expression level of a gene” as used herein refers to detecting and quantifying RNA transcribed from that gene. The term “RNA transcript” includes mRNA transcribed from the gene, and/or specific spliced variants thereof and/or fragments of such mRNA and spliced variants.

A person skilled in the art will appreciate that a number of methods can be used to isolate RNA from the tissue sample for analysis. For example, RNA may be isolated from frozen tissue samples by homogenization in guanidinium isothiocyanate and acid phenol-chloroform extraction. Commercial kits are available for isolating RNA from FFPE samples. If the tumor sample is an FFPE tissue section on a glass slide, it is possible to perform gene expression analysis on whole cell lysates rather than on isolated total RNA.

Persons skilled in the art are also aware of several methods useful for detecting and quantifying the level of RNA transcripts within the isolated RNA or whole cell lysates. Quantitative detection methods include, but are not limited to, arrays (i.e., microarrays), quantitative real time PCR (RT-PCR), multiplex assays, nuclease protection assays, and Northern blot analyses. Generally, such methods employ labeled probes that are complimentary to a portion of each transcript to be detected. Probes for use in these methods can be readily designed based on the known sequences of the genes and the transcripts expressed thereby. Suitable labels for the probes are well-known and include, e.g., fluorescent, chemiluminescent and radioactive labels.

In some embodiments, assaying a tumor sample for expression of the genes in Table 1, or gene signatures derived therefrom (i.e. gene signatures comprising 5 or more genes from Table 1), employs detection and quantification of RNA levels in real-time using nucleic acid sequence based amplification (NASBA) combined with molecular beacon detection molecules. NASBA is described, e.g., in Compton, Nature 350 (6313):91-92 (1991). NASBA is a single-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

In other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In yet other embodiments, the assay format employs direct mRNA capture with branched DNA (QuantiGene™, Panomics) or Hybrid Capture™ (Digene).

One example of an array technology suitable for use in measuring expression of the genes in gene expression platform of the invention is the ArrayPlate™ assay technology sold by HTG Molecular, Tucson Ariz., and described in Martel, R. R., et al., Assay and Drug Development Technologies 1(1):61-71, 2002. In brief, this technology combines a nuclease protection assay with array detection. Cells in microplate wells are subjected to a nuclease protection assay. Cells are lysed in the presence of probes that bind targeted mRNA species. Upon addition of SI nuclease, excess probes and unhybridized mRNA are degraded, so that only mRNA:probe duplexes remain. Alkaline hydrolysis destroys the mRNA component of the duplexes, leaving probes intact. After the addition of a neutralization solution, the contents of the processed cell culture plate are transferred to another ArrayPlate™ called a programmed ArrayPlate™. ArrayPlates™ contain a 16-element array at the bottom of each well. Each array element comprises a position-specific anchor oligonucleotide that remains the same from one assay to the next. The binding specificity of each of the 16 anchors is modified with an oligonucleotide, called a programming linker oligonucleotide, which is complementary at one end to an anchor and at the other end to a nuclease protection probe. During a hybridization reaction, probes transferred from the culture plate are captured by immobilized programming linker. Captured probes are labeled by hybridization with a detection linker oligonucleotide, which is in turn labeled with a detection conjugate that incorporates peroxidase. The enzyme is supplied with a chemiluminescent substrate, and the enzyme-produced light is captured in a digital image. Light intensity at an array element is a measure of the amount of corresponding target mRNA present in the original cells.

By way of further example, DNA microarrays can be used to measure gene expression. In brief, a DNA microarray, also referred to as a DNA chip, is a microscopic array of DNA fragments, such as synthetic oligonucleotides, disposed in a defined pattern on a solid support, wherein they are amenable to analysis by standard hybridization methods (see Schena, BioEssays 18:427 (1996)). Exemplary microarrays and methods for their manufacture and use are set forth in T. R. Hughes et al., Nature Biotechnology 9:342-347 (2001). A number of different microarray configurations and methods for their production are known to those of skill in the art and are disclosed in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 6,022,963; 6,077,674; and U.S. Pat. No. 6,156,501; Shena, et al., Tibtech 6:301-306, 1998; Duggan, et al., Nat. Genet. 2:10-14, 1999; Bowtell, et al., Nat. Genet. 21:25-32, 1999; Lipshutz, et al., Nat. Genet. 21:20-24, 1999; Blanchard, et al., Biosensors and Bioelectronics 77:687-90, 1996; Maskos, et al., Nucleic Acids Res. 2:4663-69, 1993; and Hughes, et al., Nat. Biotechnol. 79:342-347, 2001. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; the disclosures of which are herein incorporated by reference.

In one embodiment, an array of oligonucleotides may be synthesized on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as “DNA chips” or very large scale immobilized polymer arrays (“VLSIPS®” arrays), may include millions of defined probe regions on a substrate having an area of about 1 cm² to several cm², thereby incorporating from a few to millions of probes (see, e.g., U.S. Pat. No. 5,631,734).

To compare expression levels, labeled nucleic acids may be contacted with the array under conditions sufficient for binding between the target nucleic acid and the probe on the array. In one embodiment, the hybridization conditions may be selected to provide for the desired level of hybridization specificity; that is, conditions sufficient for hybridization to occur between the labeled nucleic acids and probes on the microarray.

Hybridization may be carried out in conditions permitting essentially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed.; Elsevier, N.Y. (1993)). The methods described above will result in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent Gene Array® Scanner. This scanner is controlled from a system computer with an interface and easy-to-use software tools. The output may be directly imported into or directly read by a variety of software applications. Exemplary scanning devices are described in, for example, U.S. Pat. Nos. 5,143,854 and 5,424,186.

One assay method to measure transcript abundance for the genes listed in Table 1 utilizes the nCounter® Analysis System marketed by NanoString© Technologies (Seattle, Wash. USA). This system, which is described by Geiss et al., Nature Biotechnol. 2(3):317-325 (2008), utilizes a pair of probes, namely, a capture probe and a reporter probe, each comprising a 35- to 50-base sequence complementary to the transcript to be detected. The capture probe additionally includes a short common sequence coupled to an immobilization tag, e.g. an affinity tag that allows the complex to be immobilized for data collection. The reporter probe additionally includes a detectable signal or label, e.g. is coupled to a color-coded tag. Following hybridization, excess probes are removed from the sample, and hybridized probe/target complexes are aligned and immobilized via the affinity or other tag in a cartridge. The samples are then analyzed, for example using a digital analyzer or other processor adapted for this purpose. Generally, the color-coded tag on each transcript is counted and tabulated for each target transcript to yield the expression level of each transcript in the sample. This system allows measuring the expression of hundreds of unique gene transcripts in a single multiplex assay using capture and reporter probes designed by NanoString.

In particular embodiments of the invention where the nCounter® Analysis System is used to measure RNA level of the genes in Table 1, the normalization gene set comprises 10-12 genes selected from the genes listed in Table 2.

TABLE 2
Normalization Genes Useful with nCounter ®
Analysis System
Normalization Genes

| Gene Symbol | Accession No. |
|---|---|
| ABCF1 | NM_001090.2 |
| C14ORF102 | NM_017970.3 |
| G6PD | NM_000402.2 |
| OAZ1 | NM_004152.2 |
| POLR2A | NM_000937.2 |
| SDHA | NM_004168.1 |
| STK11IP | NM_052902.2 |
| TBC1D10B | NM_015527.3 |
| TBP | NM_001172085.1 |
| UBB | NM_018955.2 |
| ZBTB34 | NM_001099270.1 |

Another tool for detecting expression of the genes in the stromal/EMT/TGFβ gene signature biomarker (i.e., the genes disclosed in Table 1) is RNA-Seq. See Wang et al., RNA-Seq: a revolutionary tool for transcriptomics. Nat Rev Genet. 10(1): 57-63 (2009); doi:10.1038/nrg2484. RNA-Seq uses deep-sequencing technologies and provides a precise measurement of levels of transcripts and their isoforms. Briefly, RNA is extracted and converted to a library of cDNA fragments with adaptors ligated to either one end or both ends. The molecules are then sequenced in a high-throughput manner to obtain short sequences using any available high throughput sequencing technology. The resulting sequence information is aligned to a reference genome or transcripts or de novo assembled into a genome-scale transcription map that comprises the level of expression for each gene.

In measuring expression of the clinical response genes in Table 1 described herein, the absolute expression of each of the genes in a tumor sample is compared to a control; for example, the control can be the average level of expression of each of the genes, respectively, in a pool of subjects. To increase the sensitivity of the comparison, however, the expression level values are preferably transformed in a number of ways.

Raw expression values of the clinical response genes in a gene expression platform described herein may be normalized by any of the following: quantile normalization to a common reference distribution, by the mean RNA levels of a set of housekeeping genes, by global normalization relying on percentile, e.g., 75^{th} percentile, or other biologically relevant normalization approaches known to those skilled in the art.

For example, the expression level of each clinical response gene can be normalized by the average RNA expression level of all of the genes in the gene expression platform, or by the average expression level of a set of normalization genes, e.g., housekeeping genes. Thus, in one embodiment, the genes in a gene expression platform are represented by a set of probes, and the RNA expression level of each of the genes is normalized by the mean or median expression level across all of the represented genes, i.e., across all clinical response and normalization genes in a gene expression platform described herein In a specific embodiment, the normalization is carried out by dividing the median or mean level of RNA expression of all of the genes in the gene expression platform. In another embodiment, the RNA expression levels of the clinical response genes are normalized by the mean or median level of expression of a set of normalization genes. In a specific embodiment, the normalization genes comprise housekeeping genes. In another specific embodiment, the normalization of a measured RNA expression level for a clinical response gene is accomplished by dividing the measured level by the median or mean expression level of the normalization genes.

The sensitivity of a gene signature score may be increased if the expression levels of individual genes in the gene signature are compared to the expression of the same genes in a pool of tumor samples. Preferably, the comparison is to the mean or median expression level of each signature gene in the pool of samples. This has the effect of accentuating the relative differences in expression between genes in the sample and genes in the pool as a whole, making comparisons more sensitive and more likely to produce meaningful results than the use of absolute expression levels alone. The expression level data may be transformed in any convenient way; preferably, the expression level data for all genes is log transformed before means or medians are taken.

In performing comparisons to a pool, two approaches may be used. First, the expression levels of the signature genes in the sample may be compared to the expression level of those genes in the pool, where nucleic acid derived from the sample and nucleic acid derived from the pool are hybridized during the course of a single experiment. Such an approach requires that a new pool of nucleic acid be generated for each comparison or limited numbers of comparisons, and is therefore limited by the amount of nucleic acid available. Alternatively, and preferably, the expression levels in a pool, whether normalized and/or transformed or not, are stored on a computer, or on computer-readable media, to be used in comparisons to the individual expression level data from the sample (i.e., single-channel data).

When comparing a subject's tumor sample with a standard or control, the expression value of a particular gene in the sample is compared to the expression value of that gene in the standard or control. For each gene in a gene signature of the invention, the log(10) ratio is created for the expression value in the individual sample relative to the standard or control. A score for a gene signature is calculated by determining the mean log(10) ratio of the genes in the signature. If the gene signature score for the test sample is equal to or greater than a pre-determined threshold for that gene signature, then the sample is considered to be positive for the gene signature biomarker. The pre-determined threshold may also be the mean, median, or a percentile of scores for that gene signature in a collection of samples or a pooled sample used as a standard or control.

It will be recognized by those skilled in the art that other differential expression values, besides log(10) ratio, may be used for calculating a signature score, as long as the value represents an objective measurement of transcript abundance of the genes. Examples include, but are not limited to: xdev, error-weighted log (ratio), and mean subtracted log(intensity).

Each of the steps of obtaining a tissue sample, preparing one or more tissue sections therefrom for assaying gene expression, performing the assay, and scoring the results may be performed by separate individuals at separate locations. For example, a surgeon may obtain by biopsy a tissue sample from a cancer patient's tumor and then send the tissue sample to a pathology lab, and a technician in the lab may fix the tissue sample and then prepare one or more slides, each with a single tissue section, for the assay. The slide(s) may be assayed soon after preparation, or stored for future assay. The lab that prepared a tissue section may conduct the assay or send the slide(s) to a different lab to conduct the assay. A technician who scores the slide(s) for a gene signature may work for the diagnostic lab, or may be an independent contractor. Alternatively, a single diagnostic lab obtains the tissue sample from the subject's physician or surgeon and then performs all of the steps involved in preparing tissue sections, assaying the slide(s) and calculating the gene signature score for the tissue section(s).

In some embodiments, the individuals involved with preparing and assaying the tissue section for a gene signature or gene signature biomarker do not know the identity of the subject whose sample is being tested; i.e., the sample received by the laboratory is made anonymous in some manner before being sent to the laboratory. For example, the sample may be merely identified by a number or some other code (a “sample ID”) and the results of the assay are reported to the party ordering the test using the sample ID. In preferred embodiments, the link between the identity of a subject and the subject's tissue sample is known only to the individual or to the individual's physician.

In some embodiments, after the test results have been obtained, the diagnostic laboratory generates a test report, which may comprise any one or both of the following results: the tissue sample was biomarker positive or negative, the gene signature score for the tumor sample and the reference score for that gene signature. The test report may also include a list of genes whose expression was analyzed in the assay.

In other embodiments, the test report may also include guidance on how to interpret the results for predicting if a subject is likely to respond to a PD-1 antagonist. For example, in one embodiment, it the tested tumor sample is from a melanoma and has a gene signature score that is at or above a prespecified threshold, the test report may indicate that the subject has a score that is associated with response or better response to treatment with the PD-1 antagonist, while if the gene signature score is below the threshold, then the test report indicates that the patient has a score that is associated with no response or poor response to treatment with the PD-1 antagonist.

In some embodiments, the test report is a written document prepared by the diagnostic laboratory and sent to the patient or the patient's physician as a hard copy or via electronic mail. In other embodiments, the test report is generated by a computer program and displayed on a video monitor in the physician's office. The test report may also comprise an oral transmission of the test results directly to the patient or the patient's physician or an authorized employee in the physician's office. Similarly, the test report may comprise a record of the test results that the physician makes in the patient's file.

Assaying tumor samples for expression of the genes in a gene expression platform or gene signature described herein may be performed using a kit that has been specially designed for this purpose. In one embodiment, the kit comprises a set of oligonucleotide probes capable of hybridizing to the genes listed in Table 1. In another embodiment, the kit comprises a set of oligonucleotide probes capable of hybridizing to the genes listed in Table 1. The set of oligonucleotide probes may comprise an ordered array of oligonucleotides on a solid surface, such as a microchip, silica beads (such as BeadArray technology from Illumina, San Diego, Calif.), or a glass slide (see, e.g., WO 98/20020 and WO 98/20019). In some embodiments, the oligonucleotide probes are provided in one or more compositions in liquid or dried form.

Oligonucleotides in kits of the invention are capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

In some embodiments, each oligonucleotide in the kit is a perfect complement of its target region. An oligonucleotide is said to be a “perfect” or “complete” complement of another nucleic acid molecule if every nucleotide of one of the molecules is complementary to the nucleotide at the corresponding position of the other molecule. While perfectly complementary oligonucleotides are preferred for detecting transcripts of the Table 1 genes, departures from complete complementarity are contemplated where such departures do not prevent the molecule from specifically hybridizing to the target region as defined above. For example, an oligonucleotide probe may have one or more non-complementary nucleotides at its 5′ end or 3′ end, with the remainder of the probe being completely complementary to the target region. Alternatively, non-complementary nucleotides may be interspersed into the probe as long as the resulting probe is still capable of specifically hybridizing to the target region.

In some preferred embodiments, each oligonucleotide in the kit specifically hybridizes to its target region under stringent hybridization conditions. Stringent hybridization conditions are sequence-dependent and vary depending on the circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium.

Typically, stringent conditions include a salt concentration of at least about 0.01 to 1.0 M sodium ion concentration (or other salts) at pH 7.0 to 8. 3 and the temperature is at least about 25° C. for short oligonucleotide probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations. Additional stringent conditions can be found in Molecular Cloning: A Laboratory Manual, Sambrook et al., Cold Spring Harbor Press, Cold Spring Harbor, N.Y. (1989), chapters 7, 9, and 11, and in NUCLEIC ACID HYBRIDIZATION, A PRACTICAL APPROACH, Haymes et al., IRL Press, Washington, D.C., 1985.

One non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Stringency conditions with ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tm is determined according to the following equations. For hybrids less than 18 base pairs in length, Tₘ (° C.)=2(# of A+T bases)+4(# of G+C bases). For hybrids between 18 and 49 base pairs in length, Tₘ (° C.)=81.5+16.6(log₁₀[Na+])+0.41(% (G+C)−(600/N), where N is the number of bases in the hybrid, and [Na+] is the concentration of sodium ions in the hybridization buffer ([Na+] for 1×SSC=0.165 M).

The oligonucleotides in kits of the invention may be comprised of any phosphorylation state of ribonucleotides, deoxyribonucleotides, and acyclic nucleotide derivatives, and other functionally equivalent derivatives. Alternatively, the oligonucleotides may have a phosphate-free backbone, which may be comprised of linkages such as carboxymethyl, acetamidate, carbamate, polyamide (peptide nucleic acid (PNA)) and the like (Varma, in MOLECULAR BIOLOGY AND BIOTECHNOLOGY, A COMPREHENSIVE DESK REFERENCE, Meyers, ed., pp. 6 17-20, VCH Publishers, Inc., 1995). The oligonucleotides may be prepared by chemical synthesis using any suitable methodology known in the art, or may be derived from a biological sample, for example, by restriction digestion. The oligonucleotides may contain a detectable label, according to any technique known in the art, including use of radiolabels, fluorescent labels, enzymatic labels, proteins, haptens, antibodies, sequence tags and the like. The oligonucleotides in the kit may be manufactured and marketed as analyte specific reagents (ASRs) or may be constitute components of an approved diagnostic device.

Kits of the invention may also contain other reagents such as hybridization buffer and reagents to detect when hybridization with a specific target molecule has occurred. Detection reagents may include biotin- or fluorescent-tagged oligonucleotides and/or an enzyme-labeled antibody and one or more substrates that generate a detectable signal when acted on by the enzyme. It will be understood by the skilled artisan that the set of oligonucleotides and reagents for performing the assay will be provided in separate receptacles placed in the kit container if appropriate to preserve biological or chemical activity and enable proper use in the assay.

In other embodiments, each of the oligonucleotide probes and all other reagents in the kit have been quality tested for optimal performance in an assay designed to quantify tumor RNA expression levels, in an FFPE tumor section, of the genes in Table 1. In some embodiments, the kit includes an instruction manual that describes how to calculate a gene signature score from the quantified RNA expression levels.

### III. Methods of Treatment of the Invention and PD-1 Antagonists Useful in Said Methods

The invention provides methods of treating cancer in a human patient comprising administering to the patient a PD-1 antagonist, wherein the patient has tested positive for a stromal/EMT/TGFβ gene signature biomarker (i.e. is a low expresser of the genes in the stromal/EMT/TGFβ gene signature). PD-1 antagonists useful in the treatment methods of the invention include anti-PD-1 antibodies, or antigen binding fragments thereof, that specifically bind to PD-1 and block binding of PD-1 to PD-L1 and/or PD-L2. Other PD-1 antagonists useful in the treatment methods of the invention include anti-PD-L1 antibodies, or antigen binding fragments thereof, that specifically bind to PD-L1 and block binding of PD-L1 to PD-1.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is pembrolizumab (KEYTRUDA™, Merck & Co., Inc., Kenilworth, N.J., USA), nivolumab (OPDIVO™, Bristol-Myers Squibb Company, Princeton, N.J., USA), atezolizumab (TECENTRIQ™, Genentech, San Francisco, Calif., USA), durvalumab (IMFINZI™, AstraZeneca Pharmaceuticals LP, Wilmington, Del.), cemiplimab (LIBTAYO™, Regeneron Pharmaceuticals, Tarrytown, N.Y., USA) or avelumab (BAVENCIO™, Merck KGaA, Darmstadt, Germany). In other embodiments, the PD-1 antagonist is pidilizumab (U.S. Pat. No. 7,332,582), AMP-514 (MedImmune LLC, Gaithersburg, Md., USA), PDR001 (U.S. Pat. No. 9,683,048), BGB-A317 (U.S. Pat. No. 8,735,553), and MGA012 (MacroGenics, Rockville, Md.).

In some embodiments, the PD-1 antagonist is the anti-human PD-1 antibody, antigen binding fragment thereof, or variant thereof disclosed in any of U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

In some embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is cemiplimab. In particular embodiments, the method comprises administering 350 mg of cempiplimab to the patient about every three weeks.

In some embodiments, the PD-1 antagonist is avelumab. In particular sub-embodiments, the method comprises administering 800 mg of avelumab to the patient about every two weeks.

Table 3 provides amino acid sequences for exemplary anti-human PD-1 antibodies pembrolizumab and nivolumab. Alternative PD-1 antibodies and antigen-binding fragments that are useful in the formulations and methods of the invention are shown in Table 4.

In some embodiments of the methods of treatment of the invention, a PD-1 antagonist is an anti-human PD-1 antibody or antigen binding fragment thereof or an anti-human PD-L1 antibody or antigen binding fragment thereof, which comprises three light chain CDRs of CDRL1, CDRL2 and CDRL3 and/or three heavy chain CDRs of CDRH1, CDRH2 and CDRH3.

In one embodiment of the methods of treatment of the invention, CDRL1 is SEQ ID NO:1 or a variant of SEQ ID NO: 1, CDRL2 is SEQ ID NO:2 or a variant of SEQ ID NO:2, and CDRL3 is SEQ ID NO:3 or a variant of SEQ ID NO: 3.

In one embodiment, CDRH1 is SEQ ID NO:6 or a variant of SEQ ID NO:6, CDRH2 is SEQ ID NO: 7 or a variant of SEQ ID NO:7, and CDRH3 is SEQ ID NO:8 or a variant of SEQ ID NO: 8.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO: 8.

In an alternative embodiment of the invention, CDRL1 is SEQ ID NO: 11 or a variant of SEQ ID NO:11, CDRL2 is SEQ ID NO:12 or a variant of SEQ ID NO:12, and CDRL3 is SEQ ID NO:13 or a variant of SEQ ID NO: 13.

In one embodiment, CDRH1 is SEQ ID NO: 16 or a variant of SEQ ID NO:16, CDRH2 is SEQ ID NO: 17 or a variant of SEQ ID NO:17, and CDRH3 is SEQ ID NO:18 or a variant of SEQ ID NO: 18.

In one embodiment, the three light chain CDRs are SEQ ID NO:1, SEQ ID NO:2, and SEQ ID NO:3 and the three heavy chain CDRs are SEQ ID NO:6, SEQ ID NO:7 and SEQ ID NO: 8.

In an alternative embodiment, the three light chain CDRs are SEQ ID NO:11, SEQ ID NO:12, and SEQ ID NO: 13 and the three heavy chain CDRs are SEQ ID NO:16, SEQ ID NO:17 and SEQ ID NO: 18.

In a further embodiment of the invention, CDRL1 is SEQ ID NO:21 or a variant of SEQ ID NO:21, CDRL2 is SEQ ID NO:22 or a variant of SEQ ID NO:22, and CDRL3 is SEQ ID NO:23 or a variant of SEQ ID NO:23.

In yet another embodiment, CDRH1 is SEQ ID NO:24 or a variant of SEQ ID NO:24, CDRH2 is SEQ ID NO: 25 or a variant of SEQ ID NO:25, and CDRH3 is SEQ ID NO:26 or a variant of SEQ ID NO:26.

In another embodiment, the three light chain CDRs are SEQ ID NO:21, SEQ ID NO:22, and SEQ ID NO:23 and the three heavy chain CDRs are SEQ ID NO:24, SEQ ID NO:25 and SEQ ID NO:26.

Some antibody and antigen binding fragments of the methods of treatment of the invention comprise a light chain variable region and a heavy chain variable region. In some embodiments, the light chain variable region comprises SEQ ID NO:4 or a variant of SEQ ID NO:4, and the heavy chain variable region comprises SEQ ID NO:9 or a variant of SEQ ID NO:9. In further embodiments, the light chain variable region comprises SEQ ID NO: 14 or a variant of SEQ ID NO:14, and the heavy chain variable region comprises SEQ ID NO:19 or a variant of SEQ ID NO: 19. In further embodiments, the heavy chain variable region comprises SEQ ID NO:27 or a variant of SEQ ID NO:27 and the light chain variable region comprises SEQ ID NO:28 or a variant of SEQ ID NO:28, SEQ ID NO:29 or a variant of SEQ ID NO:29, or SEQ ID NO:30 or a variant of SEQ ID NO:30. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the methods of treatment of the invention, the PD-1 antagonist is an antibody or antigen binding fragment that comprises a light chain variable region comprising or consisting of SEQ ID NO:4 and a heavy chain variable region comprising or consisting SEQ ID NO:9. In a further embodiment, the antibody or antigen binding fragment comprises a light chain variable region comprising or consisting of SEQ ID NO:14 and a heavy chain variable region comprising or consisting of SEQ ID NO:19. In one embodiment of the formulations of the invention, the antibody or antigen binding fragment comprises a light chain variable region comprising or consisting of SEQ ID NO:28 and a heavy chain variable region comprising or consisting SEQ ID NO:27. In a further embodiment, the antibody or antigen binding fragment comprises a light chain variable region comprising or consisting of SEQ ID NO:29 and a heavy chain variable region comprising or consisting SEQ ID NO:27. In another embodiment, the antibody or antigen binding fragment comprises a light chain variable region comprising or consisting of SEQ ID NO:30 and a heavy chain variable region comprising or consisting SEQ ID NO:27.

In another embodiment of the methods of treatment of the invention, the PD-1 antagonist is an antibody or antigen binding protein that has a VL domain and/or a V_{H} domain with at least 95%, 90%, 85%, 80%, 75% or 50% sequence homology to one of the V_{L} domains or V_{H} domains described above, and exhibits specific binding to PD-1. In another embodiment of the methods of treatment of the invention, the PD-1 antagonist is an antibody or antigen binding protein comprising V_{L} and V_{H} domains having up to 1, 2, 3, 4, or 5 or more amino acid substitutions, and exhibits specific binding to PD-1.

In any of the embodiments above, the PD-1 antagonist may be a full-length anti-PD-1 antibody or an antigen binding fragment thereof that specifically binds human PD-1, or a full-length anti-PD-L1 antibody or an antigen binding fragment thereof that specifically binds human PD-L1. In certain embodiments, the anti-PD-1 antibody or anti-PD-L1 antibody is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the methods of treatment of the invention, the PD-1 antagonist is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO:10. In alternative embodiments, the PD-1 antagonist is an anti-PD-1 antibody comprising a light chain comprising or consisting of a sequence of amino acid residues as set forth in SEQ ID NO:15 and a heavy chain comprising or consisting of a sequence of amino acid residues as set forth in SEQ ID NO:20. In further embodiments, the PD-1 antagonist is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the PD-1 antagonist is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID NO:31. In yet additional embodiments, the PD-1 antagonist is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31.

In some embodiments of the methods of treatment of the invention, the PD-1 antagonist is pembrolizumab, a pembrolizumab variant or a pembrolizumab biosimilar. In some embodiments, the PD-1 antagonist is nivolumab, a nivolumab variant or a nivolumab biosimilar. In some embodiments, the PD-1 antagonist is atezolizumab, an atezolizumab variant or an atezolizumab biosimilar. In some embodiments, the PD-1 antagonist is durvalumab, a durvalumab variant or a durvalumab biosimilar. In some embodiments, the PD-1 antagonist is cemiplimab, a cemiplimab variant or a cemiplimab biosimilar. In some embodiments, the PD-1 antagonist is avelumab, an avelumab variant or an avelumab biosimilar.

Ordinarily, amino acid sequence variants of the PD-1 antagonists useful in the methods of treatment of the invention will have an amino acid sequence having at least 75% amino acid sequence identity with the amino acid sequence of a reference antibody or antigen binding fragment (e.g. heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

TABLE 3
Exemplary Anti-PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTS | 4 |
| Region | GYSYLHWYQQKPGQAPRLLIYLASYLESGVPA | |
| | RFSGSGSGTDFTLTISSLEPEDFAVYYCQHSR | |
| | DLPLTFGGGTKVEIK | |
| Light | EIVLTQSPATLSLSPGERATLSCRASKGVSTS | 5 |
| Chain | GYSYLHWYQQKPGQAPRLLIYLASYLESGVPA | |
| | RFSGSGSGTDFTLTISSLEPEDFAVYYCQHSR | |
| | DLPLTFGGGTKVEIKRTVAAPSVFIFPPSDEQ | |
| | LKSGTASVVCLLNNFYPREAKVQWKVDNALQS | |
| | GNSQESVTEQDSKDSTYSLSSTLTLSKADYEK | |
| | HKVYACEVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNY | 9 |
| Region | YMYWVRQAPGQGLEWMGGINPSNGGTNFNEKF | |
| | KNRVTLTTDSSTTTAYMELKSLQFDDTAVYYC | |
| | ARRDYRFDMGFDYWGQGTTVTVSS | |
| Heavy | QVQLVQSGVEVKKPGASVKVSCKASGYTFTNY | 10 |
| Chain | YMYWVRQAPGQGLEWMGGINPSNGGTNFNEKF | |
| | KNRVTLTTDSSTTTAYMELKSLQFDDTAVYYC | |
| | ARRDYRFDMGFDYWGQGTTVTVSSASTKGPSV | |
| | FPLAPCSRSTSESTAALGCLVKDYFPEPVTVS | |
| | WNSGALTSGVHTFPAVLQSSGLYSLSSVVTVP | |
| | SSSLGTKTYTCNVDHKPSNTKVDKRVESKYGP | |
| | PCPPCPAPEFLGGPSVFLFPPKPKDTLMISRT | |
| | PEVTCVVVDVSQEDPEVQFNWYVDGVEVHNAK | |
| | TKPREEQFNSTYRVVSVLTVLHQDWLNGKEYK | |
| | CKVSNKGLPSSIEKTISKAKGQPREPQVYTLP | |
| | PSQEEMTKNQVSLTCLVKGFYPSDIAVEWESN | |
| | GQPENNYKTTPPVLDSDGSFFLYSRLTVDKSR | |
| | WQEGNVFSCSVMHEALHNHYTQKSLSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERATLSCRASQSVSSY | 14 |
| Region | LAWYQQKPGQAPRLLIYDASNRATGIPARFSG | |
| | SGSGTDFTLTISSLEPEDFAVYYCQQSSNWPR | |
| | TFGQGTKVEIK | |
| Light | EIVLTQSPATLSLSPGERATLSCRASQSVSSY | 15 |
| Chain | LAWYQQKPGQAPRLLIYDASNRATGIPARFSG | |
| | SGSGTDFTLTISSLEPEDFAVYYCQQSSNWPR | |
| | TFGQGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLRLDCKASGITFSNS | 19 |
| Region | GMHWVRQAPGKGLEWVAVIWYDGSKRYYADSV | |
| | KGRFTISRDNSKNTLFLQMNSLRAEDTAVYYC | |
| | ATNDDYWGQGTLVTVSS | |
| Heavy | QVQLVESGGGVVQPGRSLRLDCKASGITFSNS | 20 |
| Chain | GMHWVRQAPGKGLEWVAVIWYDGSKRYYADSV | |
| | KGRFTISRDNSKNTLFLQMNSLRAEDTAVYYC | |
| | ATNDDYWGQGTLVTVSSASTKGPSVFPLAPCS | |
| | RSTSESTAALGCLVKDYFPEPVTVSWNSGALT | |
| | SGVHTFPAVLQSSGLYSLSSVVTVPSSSLGTK | |
| | TYTCNVDHKPSNTKVDKRVESKYGPPCPPCPA | |
| | PEFLGGPSVFLFPPKPKDTLMISRTPEVTCVV | |
| | VDVSQEDPEVQFNWYVDGVEVHNAKTKPREEQ | |
| | FNSTYRVVSVLTVLHQDWLNGKEYKCKVSNKG | |
| | LPSSIEKTISKAKGQPREPQVYTLPPSQEEMT | |
| | KNQVSLTCLVKGFYPSDIAVEWESNGQPENNY | |
| | KTTPPVLDSDGSFFLYSRLTVDKSRWQEGNVF | |
| | SCSVMHEALHNHYTQKSLSLSLGK | |

TABLE 4
Additional PD-1 Antibodies and Antigen Binding Fragments Useful in the
Methods of Treatment of the Invention.
A. Antibodies and antigen binding fragments comprising light
and heavy chain CDRs of hPD-1.08A in WO2008/156712

| | |
|---|---|
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |

C. Antibodies and antigen binding fragments comprising
the mature h109A heavy chain variable region and one of the mature
K09A light chain variable regions in WO 2008/156712

| | |
|---|---|
| Heavy chain VR | SEQ ID NO: 27 |
| Light chain VR | SEQ ID NO: 28, SEQ ID NO: 29, SEQ ID NO: 30 |

D. Antibodies and antigen binding fragments comprising the mature
409 heavy chain and one of the mature
K09A light chains in WO 2008/156712

| | |
|---|---|
| Heavy chain | SEQ ID NO: 31 |
| Light chain | SEQ ID NO: 32, SEQ ID NO: 33, SEQ ID NO: 34 |

The invention provides methods of treating a patient (e.g. a human patient) with cancer comprising administering a PD-1 antagonist to the patient, wherein the patient's tumor has tested positive for the stromal/EMT/TGFβ gene signature biomarker herein, using the methods described herein.

In the methods of treatment of the invention, any PD-1 antagonist may be used, including for example, the PD-1 antagonists disclosed in this section.

In one embodiment, the invention provides a method for treating cancer in a subject having a tumor which comprises administering to the subject a PD-1 antagonist if the tumor is positive for a stromal/EMT/TGFβ gene signature biomarker, or administering to the subject a cancer treatment that does not include a PD-1 antagonist if the tumor is negative for the biomarker; wherein the determination of whether the tumor is positive or negative for the stromal/EMT/TGFβ gene signature biomarker was made using a method as described herein.

In one embodiment, the invention provides a method for treating cancer in a subject having a tumor which comprises:

(a) determining if the tumor is positive or negative for a stromal/EMT/TGFβ gene signature biomarker, wherein the determining step comprises:
(i) obtaining a sample from the subject's tumor;
(ii) sending the tumor sample to a laboratory with a request to test the sample for the presence or absence of the stromal/EMT/TGFβ gene signature biomarker; and
(iii) receiving a report from the laboratory that states whether the tumor sample is biomarker positive or biomarker negative, wherein the tumor sample is classified as biomarker positive or biomarker negative using a method according to any of the methods described herein; and

(b) administering to the subject a PD-1 antagonist if the tumor is positive for the biomarker, or administering to the subject a cancer treatment that does not include a PD-1 antagonist if the tumor is negative for the biomarker.

In another embodiment, the invention provides a method for treating cancer in a subject having a tumor which comprises:

(a) determining if the tumor is positive or negative for a stromal/EMT/TGFβ gene signature biomarker, wherein the determining step comprises:
(i) obtaining a sample from the subject's tumor;
(ii) sending the tumor sample to a laboratory with a request to generate a stromal/EMT/TGFβ gene signature score;
(iii) receiving a report from the laboratory that states the stromal/EMT/TGFβ gene signature score, wherein the stromal/EMT/TGFβ gene signature score is generated by a method comprising:
   (1) measuring the raw RNA expression level in the tumor sample for each gene in a stromal/EMT/TGFβ gene signature; wherein the stromal/EMT/TGFβ gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD;
   (2) normalizing each of the measured raw RNA expression levels; and
   (3) calculating the arithmetic mean of the normalized RNA expression levels for each of the genes to generate the score for the stromal/EMT/TGFβ gene signature;
(iv) comparing the calculated score to a reference score for the stromal/EMT/TGFβ gene signature; and
(v) classifying the tumor as biomarker positive or biomarker negative; wherein if the calculated score is equal to or less than the reference score, then the tumor is classified as biomarker positive, and if the calculated stromal/EMT/TGFβ gene signature score is greater than the reference stromal/EMT/TGFβ gene signature score, then the tumor is classified as biomarker negative; and

(b) administering to the subject a PD-1 antagonist if the tumor is positive for the biomarker, or administering to the subject a cancer treatment that does not include a PD-1 antagonist if the tumor is negative for the biomarker.

In particular embodiments of the method above, step (a)(iii)(2) comprises normalizing each of the measured raw RNA levels for each gene in the stromal/EMT/TGFβ gene signature using the measured RNA levels of a set of normalization genes.

In some embodiments, the normalization set comprises 10-12 housekeeping genes. In further embodiments, the normalization set comprises 5, 6, 7, 8, 9, 10, 11, 12, 13, 14, 15 or more housekeeping genes.

In specific embodiments, the normalization set comprises the following genes: ABCF1, C14ORF102, G6PD, OAZ1, POLR2A, SDHA, STK11IP, TBC1D10B, TBP, UBB, and ZBTB34.

In particular embodiments, the stromal/EMT/TGFβ gene signature comprises the following genes: CD 93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, and ELTD1.

The invention further provides a method for treating cancer in a subject having a tumor which comprises:

(a) determining or having determined if the tumor is positive or negative for a stromal/EMT/TGFβ gene signature biomarker using the method as described herein;

(b) determining if the tumor is positive or negative for a T-cell inflamed gene expression profile (GEP) gene signature biomarker; which step comprises:
(i) measuring the raw RNA expression level in the tumor sample for each gene in the T-cell inflamed GEP gene signature, wherein the T-cell inflamed GEP gene signature comprises 10 or more genes selected from the group consisting of: TIGIT, CD27, CD8A, PDCD1LG2, LAG3, CD274, CXCR6, CMKLR1, NKG7, CCL5, PSMB10, IDO1, CXCL9, HLA.DQA1, CD276, STAT1, HLA.DRB1, and HLA.E;
(ii) normalizing each of the measured raw RNA expression levels;
(iii) calculating the arithmetic mean of the normalized RNA expression levels for each of the genes to generate a score for the T-cell inflamed GEP gene signature; and
(iv) classifying the tumor as biomarker positive or biomarker negative; wherein if the calculated T-cell inflamed GEP score is equal to or greater than a reference T-cell inflamed GEP score, then the tumor is classified as biomarker positive, and if the calculated T-cell inflamed GEP score is less than the reference T-cell inflamed GEP score, then the tumor is classified as biomarker negative; and

(c) administering to the subject a PD-1 antagonist if the tumor is positive for the stromal/EMT/TGFβ gene signature biomarker and positive for the T-cell inflamed GEP gene signature biomarker, or administering to the subject a cancer treatment that does not include a PD-1 antagonist if the tumor is negative for the stromal/EMT/TGFβ gene signature biomarker or negative for the T-cell inflamed GEP gene signature biomarker.

In particular embodiments, the T-cell inflamed GEP gene signature comprises 10 or more, 11 or more, 12 or more, 13 or more, 14 or more, 15 or more, 16 or more, or 17 or more genes selected from the group consisting of: TIGIT, CD27, CD8A, PDCD1LG2, LAG3, CD274, CXCR6, CMKLR1, NKG7, CCL5, PSMB10, IDO1, CXCL9, HLA.DQA1, CD276, STAT1, HLA.DRB1, and HLA.E. In some embodiments, the T-cell inflamed GEP gene signature comprises each of the following genes: TIGIT, CD27, CD8A, PDCD1LG2, LAG3, CD274, CXCR6, CMKLR1, NKG7, CCL5, PSMB10, IDO1, CXCL9, HLA.DQA1, CD276, STAT1, HLA.DRB1, and HLA.E.

In specific embodiments of any of the methods of treatment disclosed herein, the PD-1 antagonist is pembrolizumab, nivolumab, atezolizumab, durvalumab, cemiplimab, or avelumab.

In one embodiment, the PD-1 antagonist is pembrolizumab or a variant of pembrolizumab.

In one embodiment, the PD-1 antagonist is nivolumab or a variant of nivolumab.

In one embodiment, the PD-1 antagonist is avelumab or a variant of avelumab.

In one embodiment, the PD-1 antagonist is durvalumab or a variant of durvalumab.

In one embodiment, the PD-1 antagonist is cemiplimab or a variant of cemiplimab.

In one embodiment, the PD-1 antagonist is atezolizumab or a variant of atezolizumab.

The method of treatment of the invention may be useful for treating cancer, wherein the cancer is melanoma, non-small cell lung cancer, small cell lung cancer, head and neck squamous cell cancer, Hodgkin lymphoma, primary mediastinal large B-cell lymphoma, urothelial carcinoma, microsatellite instability-high cancer, gastric cancer, cervical cancer, renal cell carcinoma, esophageal cancer, Merkel cell carcinoma, endometrial carcinoma, or hepatocellular carcinoma.

In particular embodiments the cancer is locally advanced or metastatic urothelial carcinoma.

### IV. Pharmaceutical Compositions, and Drug Products and Treatment Regimens

An individual to be treated by any of the methods and products described herein is a human subject diagnosed with a tumor, and a sample of the subject's tumor is available or obtainable to use in testing for the presence or absence of a gene signature biomarker derived using gene expression platform described herein.

The tumor tissue sample can be collected from a subject before and/or after exposure of the subject to one or more therapeutic treatment regimens, such as for example, a PD-1 antagonist, a chemotherapeutic agent, radiation therapy. Accordingly, tumor samples may be collected from a subject over a period of time. The tumor sample can be obtained by a variety of procedures including, but not limited to, surgical excision, aspiration or biopsy.

A physician may use a gene signature score as a guide in deciding how to treat a patient who has been diagnosed with a type of cancer that is susceptible to treatment with a PD-1 antagonist or other chemotherapeutic agent(s). Prior to initiation of treatment with the PD-1 antagonist or the other chemotherapeutic agent(s), the physician would typically order a diagnostic test to determine if a tumor tissue sample removed from the patient is positive or negative for a gene signature biomarker. However, it is envisioned that the physician could order a first or subsequent diagnostic tests at any time after the individual is administered the first dose of the PD-1 antagonist or other chemotherapeutic agent(s). In some embodiments, a physician may be considering whether to treat the patient with a pharmaceutical product that is indicated for patients whose tumor tests positive for the gene signature biomarker. For example, if the reported score is at or above a pre-specified threshold score that is associated with response or better response to treatment with a PD-1 antagonist, the patient is treated with a therapeutic regimen that includes at least the PD-1 antagonist (optionally in combination with one or more chemotherapeutic agents), and if the reported gene signature score is below a pre-specified threshold score that is associated with no response or poor response to treatment with a PD-1 antagonist, the patient is treated with a therapeutic regimen that does not include any PD-1 antagonist.

In deciding how to use the gene signature test results in treating any individual patient, the physician may also take into account other relevant circumstances, such as the stage of the cancer, weight, gender, and general condition of the patient, including inputting a combination of these factors and the gene signature biomarker test results into a model that helps guide the physician in choosing a therapy and/or treatment regimen with that therapy.

The physician may choose to treat the patient who tests biomarker positive with a combination therapy regimen that includes a PD-1 antagonist and one or more additional therapeutic agents. The additional therapeutic agent may be, e.g., a chemotherapeutic, a biotherapeutic agent (including but not limited to antibodies to VEGF, EGFR, Her2/neu, VEGF receptors, other growth factor receptors, CD20, CD40, CD-40L, GITR, CTLA-4, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammalI and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy used to treat a biomarker positive patient may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy used to treat a biomarker positive patient may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each therapeutic agent in a combination therapy used to treat a biomarker positive patient can be administered orally or parenterally, including the intravenous, intramuscular, intraperitoneal, subcutaneous, rectal, topical, and transdermal routes of administration.

A patient may be administered a PD-1 antagonist prior to or following surgery to remove a tumor and may be used prior to, during or after radiation therapy.

In some embodiments, a PD-1 antagonist is administered to a patient who has not been previously treated with a biotherapeutic or chemotherapeutic agent, i.e., is treatment-naive. In other embodiments, the PD-1 antagonist is administered to a patient who failed to achieve a sustained response after prior therapy with a biotherapeutic or chemotherapeutic agent, i.e., is treatment-experienced.

A therapy comprising a PD-1 antagonist is typically used to treat a tumor that is large enough to be found by palpation or by imaging techniques well known in the art, such as MRI, ultrasound, or CAT scan. In some preferred embodiments, the therapy is used to treat an advanced stage tumor having dimensions of at least about 200 mm³, 300 mm³, 400 mm³, 500 mm³, 750 mm³, or up to 1000 mm³.

Selecting a dosage regimen (also referred to herein as an administration regimen) for a therapy comprising a PD-1 antagonist depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of the PD-1 antagonist that is delivered to the patient consistent with an acceptable level of side effects. Accordingly, the dose amount and dosing frequency depends in part on the particular PD-1 antagonist, any other therapeutic agents to be used, and the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents used in combination with a PD-1 antagonist may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

In certain embodiments, a subject will be administered an intravenous (IV) infusion of a medicament comprising any of the PD-1 antagonists described herein, and such administration may be part of a treatment regimen employing the PD-1 antagonist as a monotherapy regimen or as part of a combination therapy.

In another preferred embodiment of the invention, the PD-1 antagonist is pembrolizumab, which is administered in a liquid medicament at a dose selected from the group consisting of 200 mg Q3W, 400 mg Q6W, 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, and 10 mg/kg Q3W or equivalents of any of these doses. In some particularly preferred embodiments, pembrolizumab is administered as a liquid medicament which comprises 25 mg/ml pembrolizumab, 7% (w/v) sucrose, 0.02% (w/v) polysorbate 80 in 10 mM histidine buffer pH 5.5, and the selected dose of the medicament is administered by IV infusion over a time period of 30 minutes. The optimal dose for pembrolizumab in combination with any other therapeutic agent may be identified by dose escalation.

The present invention also provides a medicament which comprises a PD-1 antagonist as described above and a pharmaceutically acceptable excipient. When the PD-1 antagonist is a biotherapeutic agent, e.g., a mAb, the antagonist may be produced in CHO cells using conventional cell culture and recovery/purification technologies.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab, which are suitable for use in the present invention. In some preferred embodiments, a medicament comprising pembrolizumab is provided in a glass vial which contains about 50 mg of pembrolizumab.

These and other aspects of the invention, including the exemplary specific embodiments listed below, will be apparent from the teachings contained herein.

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

### Study Design

A gene expression signature representing convergent biology related to stromal/epithelial to mesenchymal transition (EMT)/TGF-β pathways was developed and tested for its association with response to pembrolizumab in patients with advanced urothelial cancer who participated in the KEYNOTE-052 study. KEYNOTE-052 was a single-arm phase 2 trial of pembrolizumab in cisplatin-ineligible patients with advanced urothelial carcinoma (N=370) who had not been previously treated with systemic chemotherapy (Balar, et al. First-line pembrolizumab in cisplatin-ineligible patients with locally advanced and unresectable or metastatic urothelial cancer (KEYNOTE-052): a multicentre, single-arm, phase 2 study. Lancet Oncol. 18:1483-1492 (2017)). Patients received 200 mg pembrolizumab via intravenous infusion every 3 weeks until confirmed disease progression, unacceptable toxicity, withdrawal per physician/patient decision, or completion of 2 years of treatment. The primary endpoint was objective response (the proportion of patients who achieved complete or partial response) in all patients and by PD-L1 expression status according to the Response Evaluation Criteria in Solid Tumors, version 1.1, as assessed by independent central review. PD-L1 expression was assessed in tumor and inflammatory cells. Response and safety were analyzed in all patients who received at least one dose of pembrolizumab (all-patients-treated population).

Profiling was performed using RNA-seq from the transcriptome of archived FFPE for primary tumors (N=187). See EXAMPLE 3. Baseline characteristics of patients were comparable between the total population and the subgroup of patients whose tumors underwent RNASeq analysis. See Table 5.

TABLE 5
Patient Baseline Characteristics

| | | |
|---|---|---|
| | Total Population | RNASeq Subgroup |
| Characteristic, n (%) | N = 370 | n = 187 |

Sex, n (%)

| | | |
|---|---|---|
| Male | 287 (77) | 143 (77) |
| Female | 184 (23) | 44 (23) |

Age, n (%)

| | | |
|---|---|---|
| <65 y | 68 (18) | 31 (17) |
| ≥65 y | 302 (82) | 156 (83) |

ECOG performance status, n (%)

| | | |
|---|---|---|
| 0 | 214 (58) | 104 (56) |
| 1 | 156 (42) | 83 (44) |

PD-L1 status, n (%)

| | | |
|---|---|---|
| CPS <10 | 251 (68) | 124 (67) |
| CPS ≥10 | 110 (30) | 60 (32) |
| Missing data | 9 (2) | 2 (2) |

Response, n (%)

| | | |
|---|---|---|
| No (0) | 262 (71) | 131 (70) |
| Yes (1) | 108 (29) | 56 (30) |
| Median PFS, mo (95% CI) | 2.3 (2.1-3.4) | 2.8 (2.1-3.6) |
| Median OS, mo (95% CI) | 11.0 (10.1-14.0) | 11.6 (10.6-18.5) |

BOR, best overall response; CPS, combined positive score; ECOG, Eastern Cooperative Oncology Group; NR, not reached; OS, overall survival; PD-L1, programmed death ligand-1; PFS, progression-free survival

### Example 2

### Development of a Stromal/EMT/TGF-β Signature

Several signatures indicative of high stromal content have been proposed to be associated with activation of TGFβ 3 pathway, infiltration of cancer associated fibroblasts and upregulation of EMT signature. (Yoshihara, et al. Nat Commun. 4:2612 (2013), DOI: 10.1038/ncomms3612; Loboda, et al. BMC Med Genomics, EMT is the dominant program in human colon cancer 4:9 (2011)). These signatures were previously found to be associated 10 with activation of TGF-03 pathway, infiltration of cancer associated fibroblasts and upregulation of epithelial to mesenchymal transition signature. Immunosuppressive stroma signaling may be associated with impaired response to anti-PD(L) (Wang et al., EMT- and stroma-related gene expression and resistance to PD-1 blockade in urothelial cancer. Nat Commun. 9(1):3503 (2018); Mariathasan et al. TGFβ 3 attenuates tumor response to PD-L1 blockade by contributing to exclusion of T cells. Nature 554:544-548).

Signatures indicative of high stromal content (“reference signatures”) were evaluated in the Merck-Moffitt/The Cancer Genome Atlas (“TCGA”) datasets. High pairwise correlation (>0.9) between the signature scores was observed (Ayers et al., Molecular Profiling of Cohorts of Tumor Samples to Guide Clinical Development of Pembrolizumab as Monotherapy. Clin Cancer Res. doi: 10.1158/1078-0432.CCR-18-1316 (2018)). Using these resources, we computed the correlation of all the genes with the reference signature using data in both Mofflitt and TCGA. Genes that had a difference in the individual MoffitI and TCGA correlations above 0.2 were removed. The Moffit and TCGA correlations were averaged for all genes and the consensus signature set was selected with a high correlation between the signature scores (above 0.9 for pairwise correlation). This resulted in a 51-gene consensus stromal/EMT/TGFβ 3 gene set, prespecified for analysis in KEYNOTE-052 prior to merging with clinical outcomes (See Table 1). The stromal/EMT/TGFβ signature represents a key biological axis of gene expression distinct from the GEP (Table 1). 49 of the 51 pre-specified signature genes were found to pass quality control criteria for this RNASeq data set and used to calculate the signature score (see Table 6).

TABLE 6
Gene Identifications for 18-Gene T-Cell Inflamed GEP
and Stroma/EMT/TGFβ Signature
Individual Genes

| GEP Signature | Stroma/EMT/TGFβ Signature | |
|---|---|---|
| TIGIT | CD93 | AEBP1 |
| CD27 | CDH11 | COL1A2 |
| CD8A | ECM2 | COL5A2 |
| PDCD1LG2 | PDGFRB | CD248 |
| LAG3 | GGT5 | MSRB3 |
| CD274 | THBS2 | GLT8D2 |
| CXCR6 | LRRC32 | OLFML1 |
| CMKLR1 | COL3A1 | ANGPTL2 |
| NKG7 | DCN | HEG1 |
| CCL5 | GPR124 | ADAMTS2 |
| PSMB10 | THY1 | CRISPLD2 |
| IDO1 | WISP1 | COL15A1 |
| CXCL9 | ANTXR1 | COL6A2 |
| HLA.QDA1 | COL8A1 | NID2 |
| CD276 | PCOLCE | AXL |
| STAT1 | PODN | FBN1 |
| HLA.DRB1 | ITGA11 | OLFML2B |
| HLA.E | COL5A1 | EDNRA |
| | LAMA4 | CCDC80 |
| | VCAN | NIXRA8 |
| | SPARC | TSHZ3 |
| | RUNX1T1 | FSTL1 |
| | MMP2 | HSPA12B |
| | COL6A3 | ELTD1 |
| | FILIP1L | |

### Example 3

### Statistical Analysis and Results

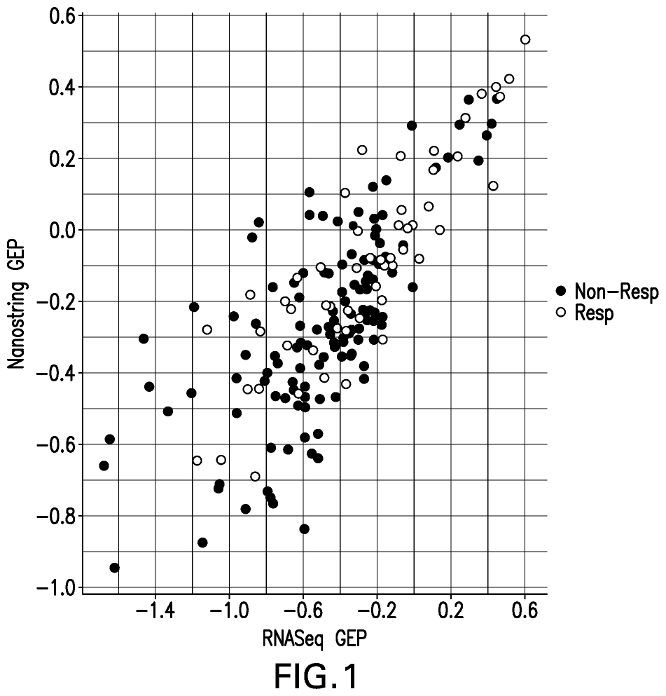

RNA-Seq-based data was available for tumors from 187 patients. RNA-seq was performed using the Illumina Hi Seq4000 platform and analyzed by BGI Americas Corporation (Cambridge Mass.). The 18-gene T-cell inflamed GEP was assessed in tumor specimens of patients with multiple tumor types treated with pembrolizumab across clinical trials (O'Donnell et al., J. Clin. Oncol. 35:4502 (2017); Haddad et al., J. Clin. Oncol. 35: 6009 (2017)). A GEP score was calculated as a weighted sum of normalized expression values of 18 genes (see Table 6), regardless of platform. The GEP (NanoString) cutoff of −0.318, previously determined in analyses that correlated objective response to pembrolizumab, was used to define a GEP non-low group by mapping to the RNASeq quantile equivalent to the quantile of KEYNOTE-052 occupied by the Nanostring −0.318 cut-off (in patients with both types of gene expression data). Among patients with both RNASeq data and earlier evaluation of the 18-gene T-cell inflamed GEP via Nanostring platform, the GEP constructed via RNASeq was strongly correlated with the Nanostring-based GEP (correlation was 0.78). The RNASeq-based GEP cut off mapping to the Nanostring cut-off of −0.318 was determined to be −0.587 (see FIG. 1).

Logistic regression models were used to assess the statistical significance (using one-sided p-values in the direction of putative resistance) of the association of the stromal/EMT/TGFβ signature score with objective response via RECIST 1.1, including evaluation of the gene signature after adjusting for the explanatory value of the GEP. ROC curves were used as a general measure of the discriminatory value of the gene signature. Models also adjusted for ECOG performance status.

Scatterplots showing the joint pattern of expression between the 18-gene T-cell inflamed GEP and the Stromal/EMT/TGFβ are provided (with GEP low and non-low cutoff noted), as well as boxplots of the Stromal/EMT/TGFβ distribution for responders vs. non-responders over the tertiles of the GEP. The robustness of the stromal/EMT/TGF-0 signature findings was assessed across tertile levels of the GEP.

The primary end points were best overall response (BOR): 1=CR/PR; 0 otherwise. Cox regression models were used to evaluate progression-free survival (PFS) with the same model terms described for BOR.

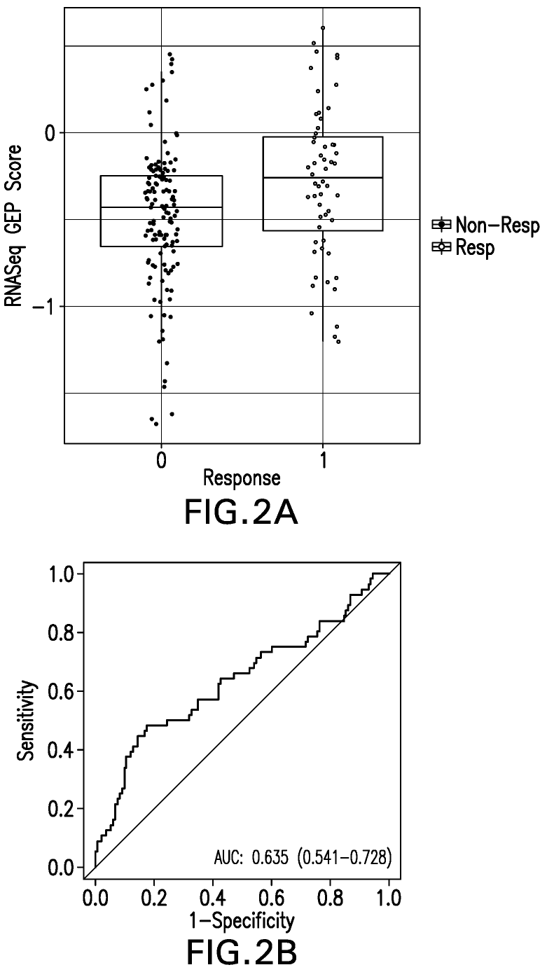

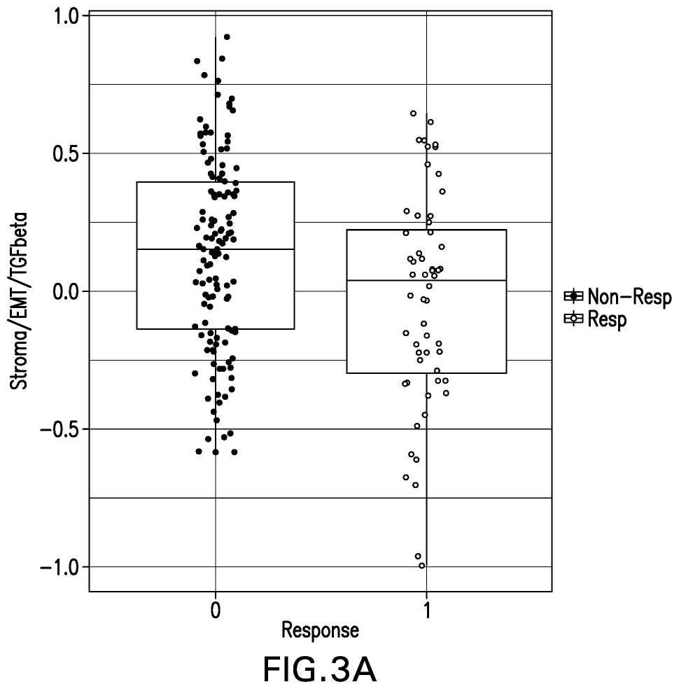

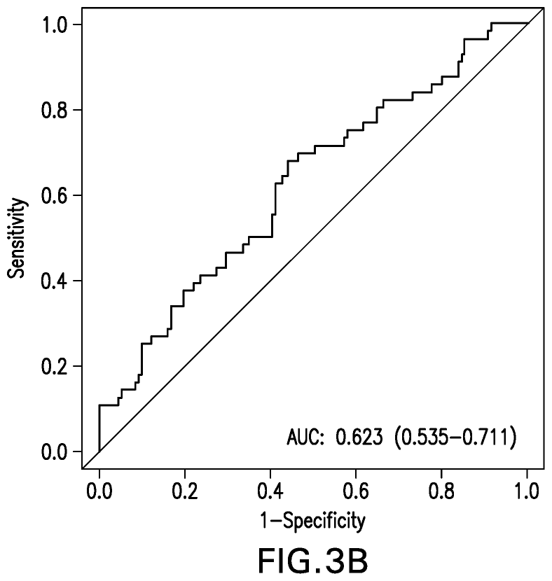

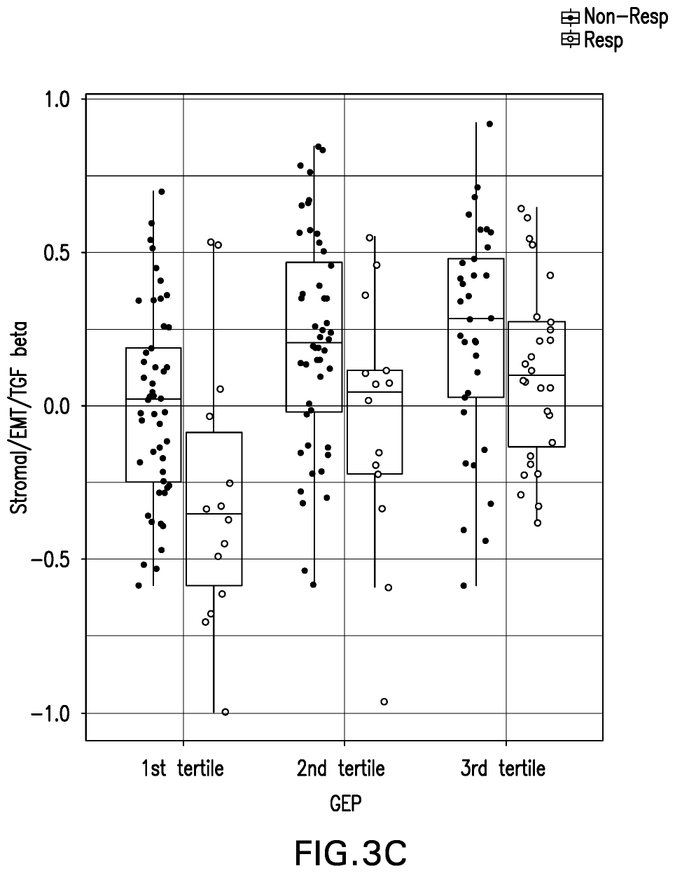

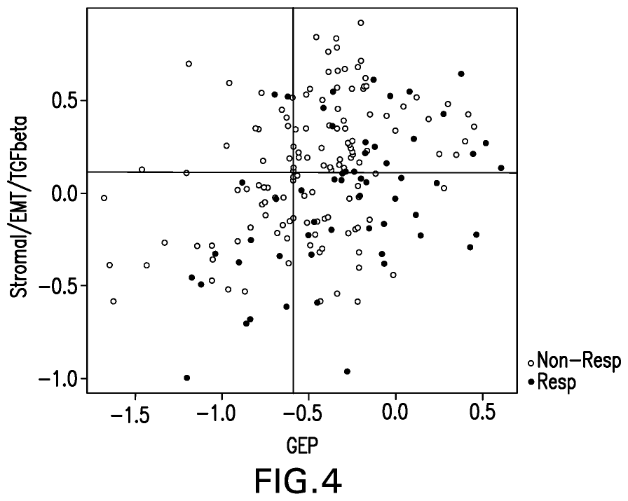

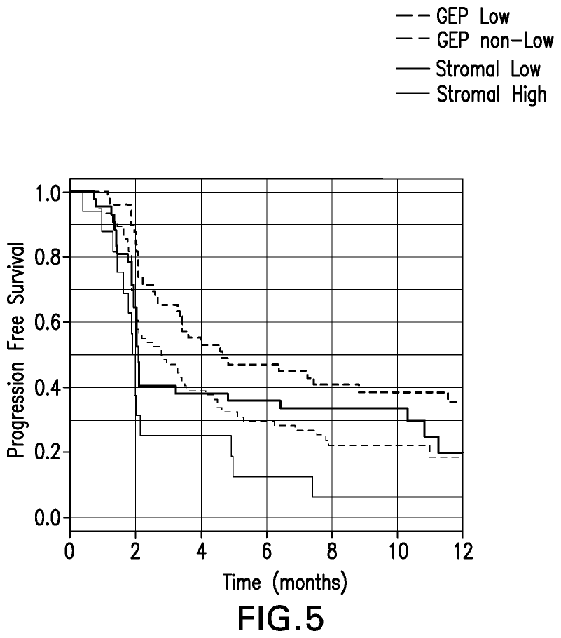

Associations of RNASeq-Based 18-Gene T-Cell Inflamed GEP with Clinical Response Results indicate that higher RNASeq GEP score (adjusting for ECOG) was associated with improved BOR (P=0.002, one-sided from logistic regression) (FIG. 2A). The AUC of ROC curve was 0.635 (CI, 0.541-0.728) (FIG. 2B).
Associations of Stromal/EMT TGFβ with Clinical Response
Lower Stromal/EMT/TGFβ score was associated with favorable BOR, regardless of GEP status (one-sided P=0.002 adjusting for ECOG PS; P<0.001 adjusting for ECOG PS and GEP) (FIG. 3A).
The AUC of ROC curve was 0.623 (CI: 0.535-0.711) (FIG. 3B).
Lower Stromal/EMT/TGFβ scores for responders to pembrolizumab were consistently observed when assessed across the tertiles of the GEP (FIG. 3C).
The highest response rate was observed in stromal/EMT/TGFβ low and GEP non-low patients, 48.0% (24 of 50 patients) (see FIG. 4).
The lowest response rate was observed in stromal/EMT/TGFβ high and GEP low patients, 11.1% (2 of 18 patients).
Similar to observations on BOR, longer estimated PFS was observed for patients with low vs high Stromal/EMT/TGFβ signature (within both low and non-low categories for the GEP) see (FIG. 5).
   PFS was significantly associated with Stromal/EMT/TGFβ signature, (one-sided P=0.007 adjusting for ECOG PS; P<0.001 adjusting for ECOG PS and GEP).
   Higher RNASeq GEP score was significantly associated with longer PFS (P=0.004).

### CONCLUSIONS

Findings related to the 18-gene T-cell inflamed GEP using RNASeq to assess expression were consistent with those using NanoString platform to correlate clinical response to pembrolizumab in the KEYNOTE-052 study (O'Donnell P., et al., J. Clin. Oncol. 2017; 35: 4502).
Higher RNASeq 18-gene T-cell inflamed GEP score was significantly associated with improved BOR (P=0.002) and PFS (P=0.004) among patients treated with pembrolizumab in the KENOTE-052 study
Lower Stromal/EMT/TGF-β score was associated with favorable BOR rate (P<0.001) and PFS (P<0.001), independently of GEP
The patterns indicated a consistent downward trend in the distribution of the Stromal/EMT/TGF-β score for pembrolizumab responders vs non-responders, regardless of GEP

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A method for testing a tumor for the presence or absence of a biomarker that predicts response to treatment with a PD-1 antagonist, which comprises:
(a) obtaining a sample from the tumor,
(b) measuring the raw RNA expression level in the tumor sample for each gene in a stromal/EMT/TGFβ gene signature, wherein the stromal/EMT/TGFβ gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD1;
(c) normalizing each of the measured raw RNA expression levels;
(d) calculating the arithmetic mean of the normalized RNA expression levels for each of the genes to generate a score for the stromal/EMT/TGFβ gene signature;
(e) comparing the calculated score to a reference score for the stromal/EMT/TGFβ gene signature; and
(f) classifying the tumor as biomarker positive or biomarker negative; wherein if the calculated score is equal to or less than the reference score, then the tumor is classified as biomarker positive, and if the calculated stromal/EMT/TGFβ gene signature score is greater than the reference stromal/EMT/TGFβ gene signature score, then the tumor is classified as biomarker negative.

2. The method of claim 1, wherein step (b) comprises normalizing each of the measured raw RNA levels for each gene in the stromal/EMT/TGFβ gene signature using the measured RNA levels of a set of normalization genes.

3. The method of claim 2, wherein the set of normalization genes comprises 10-12 housekeeping genes.

4. The method of claim 3, wherein the set of normalization genes comprises at least ten of the following genes: ABCF1, C14ORF102, G6PD, OAZ1, POLR2A, SDHA, STK11IP, TBC1D10B, TBP, UBB, and ZBTB34.

5. A method for treating cancer in a subject having a tumor which comprises administering to the subject a PD-1 antagonist if the tumor is positive for a stromal/EMT/TGFβ gene signature biomarker, or administering to the subject a cancer treatment that does not include a PD-1 antagonist if the tumor is negative for the biomarker; wherein the determination of whether the tumor is positive or negative for the stromal/EMT/TGFβ gene signature biomarker was made using a method according to claim 1.

6. A method for treating cancer in a subject having a tumor which comprises:
(a) determining if the tumor is positive or negative for a stromal/EMT/TGFβ gene signature biomarker, wherein the determining step comprises:
(i) obtaining a sample from the subject's tumor;
(ii) sending the tumor sample to a laboratory with a request to test the sample for the presence or absence of the stromal/EMT/TGFβ gene signature biomarker; and
(iii) receiving a report from the laboratory that states whether the tumor sample is biomarker positive or biomarker negative, wherein the tumor sample is classified as biomarker positive or biomarker negative using a method according to claim 1; and
(b) administering to the subject a PD-1 antagonist if the tumor is positive for the biomarker, or administering to the subject a cancer treatment that does not include a PD-1 antagonist if the tumor is negative for the biomarker.

7. A method for treating cancer in a subject having a tumor which comprises:
(a) determining if the tumor is positive or negative for a stromal/EMT/TGFβ gene signature biomarker, wherein the determining step comprises:
(i) obtaining a sample from the subject's tumor;
(ii) sending the tumor sample to a laboratory with a request to generate a stromal/EMT/TGFβ gene signature score;
(iii) receiving a report from the laboratory that states the stromal/EMT/TGFβ gene signature score, wherein the stromal/EMT/TGFβ gene signature score is generated by a method comprising:
(1) measuring the raw RNA expression level in the tumor sample for each gene in a stromal/EMT/TGFβ gene signature; wherein the stromal/EMT/TGFβ gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD;
(2) normalizing each of the measured raw RNA expression levels; and
(3) calculating the arithmetic mean of the normalized RNA expression levels for each of the genes to generate the score for the stromal/EMT/TGFβ gene signature;
(iv) comparing the calculated score to a reference score for the stromal/EMT/TGFβ gene signature; and
(v) classifying the tumor as biomarker positive or biomarker negative; wherein if the calculated score is equal to or less than the reference score, then the tumor is classified as biomarker positive, and if the calculated stromal/EMT/TGFβ gene signature score is greater than the reference stromal/EMT/TGFβ gene signature score, then the tumor is classified as biomarker negative; and
(b) administering to the subject a PD-1 antagonist if the tumor is positive for the biomarker, or administering to the subject a cancer treatment that does not include a PD-1 antagonist if the tumor is negative for the biomarker.

8. The method of claim 7, wherein step (a)(iii)(2) comprises normalizing each of the measured raw RNA levels for each gene in the stromal/EMT/TGFβ gene signature using the measured RNA levels of a set of normalization genes.

9. The method of claim 8, wherein the normalization set comprises 10-12 housekeeping genes.

10. The method of claim 9, wherein the normalization set comprises at least 10 of the following genes: ABCF1, C14ORF102, G6PD, OAZ1, POLR2A, SDHA, STK11IP, TBC1D10B, TBP, UBB, and ZBTB34.

11. The method of claim 1, wherein the stromal/EMT/TGFβ gene signature comprises the following genes: CD 93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, and ELTD1.

12. A method for treating cancer in a subject having a tumor which comprises:
(a) determining or having determined if the tumor is positive or negative for a stromal/EMT/TGFβ gene signature biomarker using the method according to claim 1;
(b) determining or having determined if the tumor is positive or negative for a T-cell inflamed gene expression profile (GEP) gene signature biomarker; which step comprises:
(i) measuring the raw RNA expression level in the tumor sample for each gene in the T-cell inflamed GEP gene signature; wherein the T-cell inflamed GEP gene signature comprises 10 or more genes selected from the group consisting of: TIGIT, CD27, CD8A, PDCD1LG2, LAG3, CD274, CXCR6, CMKLR1, NKG7, CCL5, PSMB10, IDO1, CXCL9, HLA.DQA1, CD276, STAT1, HLA.DRB1, and HLA.E;
(ii) normalizing each of the measured raw RNA expression levels;
(iii) calculating the arithmetic mean of the normalized RNA expression levels for each of the genes to generate a score for the T-cell inflamed GEP gene signature; and
(iv) classifying the tumor as biomarker positive or biomarker negative; wherein if the calculated T-cell inflamed GEP score is equal to or greater than a reference T-cell inflamed GEP score, then the tumor is classified as biomarker positive, and if the calculated T-cell inflamed GEP score is less than the reference T-cell inflamed GEP score, then the tumor is classified as biomarker negative; and
(c) administering to the subject a PD-1 antagonist if the tumor is positive for the stromal/EMT/TGFβ gene signature biomarker and positive for the T-cell inflamed GEP gene signature biomarker, or administering to the subject a cancer treatment that does not include a PD-1 antagonist if the tumor is negative for the stromal/EMT/TGFβ gene signature biomarker or negative for the T-cell inflamed GEP gene signature biomarker.

13. The method of claim 5, wherein the PD-1 antagonist is pembrolizumab, nivolumab, atezolizumab, durvalumab, cemiplimab, or avelumab.

14. The method of claim 5, wherein the PD-1 antagonist is pembrolizumab or a variant of pembrolizumab.

15. A pharmaceutical composition comprising a PD-1 antagonist for use in a subject who has a tumor that tests positive for a stromal/EMT/TGFβ gene signature biomarker, wherein the stromal/EMT/TGFβ 1 gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD1.

16. A drug product which comprises a pharmaceutical composition and prescribing information, wherein the pharmaceutical composition comprises a PD-1 antagonist and at least one pharmaceutically acceptable excipient and the prescribing information states that the pharmaceutical composition is indicated for use in a subject who has a tumor that tests positive for a stromal/EMT/TGFβ gene signature gene signature biomarker, wherein the stromal/EMT/TGFβ 1 gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD1.

17. The pharmaceutical composition of claim 15, wherein the positive biomarker test result was generated by a method according to claim 1.

18. A kit for assaying a tumor sample to determine a stromal/EMT/TGFβ gene signature score for the tumor sample, wherein the kit comprises a set of probes for detecting expression of each gene in the stromal/EMT/TGFβ gene signature, wherein the stromal/EMT/TGFβ gene signature comprises at least ten genes selected from the group consisting of: CD93, AEBP1, CDH11, COL1A2, COL5A2, ECM2, PDGFRB, CD248, GGT5, MSRB3, THBS2, GLT8D2, LRRC32, OLFML1, COL3A1, ANGPTL2, DCN, HEG1, GPR124, ADAMTS2, THY1, CRISPLD2, WISP1, COL15A1, ANTXR1, COL6A2, COL8A1, NID2, PCOLCE, AXL, PODN, FBN1, ITGA11, OLFML2B, COL5A1, EDNRA, LAMA4, CCDC80, VCAN, MXRA8, SPARC, TSHZ3, RUNX1T1, FSTL1, MMP2, HSPA12B, COL6A3, KIAA1462, FAM26E, FILIP1L, and ELTD1.

19. The method of claim 5, wherein the cancer is melanoma, non-small cell lung cancer, small cell lung cancer, head and neck squamous cell cancer, Hodgkin lymphoma, primary mediastinal large B-cell lymphoma, urothelial carcinoma, microsatellite instability-high cancer, gastric cancer, cervical cancer, renal cell carcinoma, esophageal cancer, Merkel cell carcinoma, endometrial cancer, or hepatocellular carcinoma.

20. The method of claim 5, wherein the cancer is locally advanced or metastatic urothelial carcinoma.
